(19) United States
(12) Patent Application Publication
(10) Pub. No.: US 2025/0224401 A1
(43) Pub. Date: Jul. 10, 2025

(54) METHODS OF DIAGNOSING B CELL MALIGNANCIES, DETECTING B CELL MALIGNANCY RELAPSE, AND TREATING THEREOF

(71) Applicant: Rutgers, The State University of New Jersey, New Brunswick, NJ (US)

(72) Inventors: Ping Xie, New Brunswick, NJ (US); Jaeyong Jung, New Brunswick, NJ (US); Amer Assal, New Brunswick, NJ (US); Lauri Goodell, New Brunswick, NJ (US); Roger Strair, New Brunswick, NJ (US); Samantha Gokhale, New Brunswick, NJ (US)

(21) Appl. No.: 19/094,061

(22) Filed: Mar. 28, 2025

Related U.S. Application Data
(63) Continuation of application No. PCT/US2023/033936, filed on Sep. 28, 2023.
(60) Provisional application No. 63/411,359, filed on Sep. 29, 2022.

(51) Int. Cl. G01N 33/574; A61K 31/43; A61K 31/505; A61K 31/635; A61P 31/04; G01N 33/569

(52) U.S. Cl. CPC G01N 33/57484; A61K 31/43; A61K 31/505; A61K 31/635; A61P 31/04; G01N 33/56911; G01N 2469/20

(57) ABSTRACT

Embodiments described here are directed to novel methods for detecting commensal bacteria-specific antibodies, such as commensal bacteria-specific immunoglobulin antibodies, in a blood sample or tissue fluid from a subject suspected of or suffering from a B cell malignancy, where the methods are for early detection or diagnosis of B cell malignancies and for early detection of relapse or recurrence of a B cell malignancy in remission patients previously diagnosed as suffering from a B cell malignancy. Also, described are embodiments directed to apparatuses for use with the novel methods described here. Methods of treating a subject suspected of or suffering from a B cell malignancy or a relapse or a recurrence of a B cell malignancy that can be used in combination with or without the methods of detecting commensal bacteria-specific antibodies and/or standard of care treatment are also provided here.

### CROSS REFERENCE TO RELATED APPLICATIONS

This application is a Continuation Application relating to and claiming the benefit of commonly-owned, co-pending PCT International Application No. PCT/US2023/033936, filed Sep. 28, 2023, which claims priority to and the benefit of U.S. Provisional Patent Application No. 63/411,359, filed Sep. 29, 2022, the content of each of the foregoing are herein incorporated by reference in their entirety.

### FIELD OF DISCLOSURE

The present disclosure in various embodiments described here is directed to methods and apparatuses for diagnosing cancer, such as B cell malignancies, by detecting the presence of commensal bacteria-specific antibodies in a subject sample.

### BACKGROUND

B cell malignancies include non-Hodgkin lymphomas (NHLs), Hodgkin lymphoma (HL) and multiple myeloma (MM). Non-Hodgkin lymphoma is fairly common in the United States, representing an estimated 4.2% of new cancer cases (in 2022), behind female breast cancer, male prostate cancer, lung and bronchus cancer, colorectal cancer, melanoma of the skin, and bladder cancer. NHL is the eighth leading cause of cancer death in the United States, where the death rate is 5.1 per 100,000 men and women per year (based on 2016-2020 deaths). (SEER Cancer Stat Facts: Non-Hodgkin Lymphoma. National Cancer Institute. Bethesda, MD, https://seer.cancer.gov/statfacts/html/nhl.html).

The standard diagnostic approaches to mature B cell malignancies include modern imaging technologies of patients as well as cellular and molecular diagnostic analyses of tumor/bone marrow biopsy and blood samples. Imaging technologies, including computational tomography (CT), nuclear magnetic resonance imaging (NMR or MRI), multinuclear magnetic resonance spectroscopy (MRS), positron emission tomography (PET)/CT, and PET/MRI of patients, all require the presence of adequate sizes of tumors in patients. Existing cellular and molecular diagnostic approaches include flow cytometry (FACS analysis) of B cell subpopulations, immunohistochemical (IHC) staining, cytogenetic techniques, and Ig gene clonality analysis by polymerase chain reaction (PCR) or sequencing. There are also some B cell leukemia/lymphoma 2 ELISA Kits commercially available; however, these primarily target BCL2 apoptosis regulators of the subjects. Unfortunately, the sensitivity of all these existing cellular and molecular diagnostic technologies, which depend on the number of malignant B cells present in the blood and more often in tumor/bone marrow biopsy samples, is limited by the delay necessary for malignant cell accumulation to a detectable level. The sensitivity of available methods to detect the relapse of B cell malignancies is similarly limited, which delays the detection and thus misses critical timing for effective therapy in subjects who may have a relapse or recurrence of a B cell malignancy. Accordingly, there is a pressing need for a more sensitive assay for early detection and diagnosis of B cell malignancies as well as potential relapse of B cell malignancies in an individual.

### SUMMARY

In accordance with the objectives disclosed herein, embodiments of the present disclosure satisfy the aforementioned needs and provide related advantages as well. The present disclosure provides in some objects, methods and apparatuses defined by embodiments of the disclosure and in connection with examples provided here. Other features and advantages of various embodiments of the disclosure will be apparent from the description.

As described here, the present disclosure features methods or uses comprising detecting at least one commensal bacteria-specific antibody in a sample from a subject suspected of or suffering from a B cell malignancy or a relapse or recurrence of a B cell malignancy, methods of diagnosing a B cell malignancy in a sample from the subject, methods of detecting relapse or recurrence of a B cell malignancy in a remission subject previously diagnosed as suffering from a B cell malignancy, methods of treating a subject suffering from a B cell malignancy or relapse or recurrence of a B cell malignancy, and apparatuses comprising at least one commensal bacteria-derived antigen for use in the methods disclosed herein.

The inventive disclosure is based, at least in part, on the discovery that depletion of commensal bacteria with antibiotics prevented or substantially inhibited B cell malignancy development and progression. Accordingly, antigens and/or ligands derived from commensal bacteria can be a major driver of B cell malignancy development and relapse or recurrence of a B cell malignancy. Moreover, samples (e.g., blood, plasma, serum, ascites, pleural fluid, tissue fluid or institial fluid) from a subject suffering from a B cell malignancy (e.g., lymphomas, leukemias, precursor diseases of B cell malignancies) contain antibodies (e.g., immunoglobulins (Igs)) against commensal bacterial antigens, such as proteins, glycoproteins or lipoproteins, etc. Methods of the disclosure include Western blot analyses and ELISA methods to detect commensal bacteria-specific antibodies in a blood sample for early detection of B cell malignancies and especially for early detection of relapse or recurrence in subjects who previously received B cell malignancy therapy and were in remission (i.e., partial, complete). Accordingly, antibiotics, such as bactericidal antibiotics, against commensal bacteria, are an effective treatment of B cell malignancies and prevention of or reducing likelihood of relapse.

### BRIEF DESCRIPTION OF FIGURES

FIG. 1 illustrates representative Western blots demonstrating that serum samples of aging myeloid cell-specific Traf3-deficient (M-Traf3-/-) mice with spontaneous B lymphomas (diffuse large B-cell lymphoma (DLBCL) or follicular lymphoma (FL)) contained high titers of antibodies specific for commensal bacterial proteins. Bacterial proteins were probed with serum samples at 1:500 (the left panel), 1:5000 (the middle panel), or 1:50000 (the right panel) dilutions. Symbols marked on the immunoblots: * (blue asterisk), mouse Ig light chain internal control (at ˜25 kDa in the proteins of pooled intestinal commensal bacteria harvested from the small intestines (SI) and colons of M-Traf3-/- and TRAF3-sufficient littermate control (LMC) mice, which include endogenous IgA that coated on intestinal commensal bacteria and were directly detected by the HRP-conjugated goat anti-mouse IgG (H+L) secondary (2°) antibody (Ab) for all dilutions of LMC and M-Traf3-/- #1-#3 serum samples); * (red asterisk), bacterial protein antigens specifically detected by serum Abs of M-Traf3-/- mice with B lymphomas (DLBCL or FL) (E. coli (both DH5α and Stbl3) for all dilutions of M-Traf3-/- #1-#3; K. pneumoniae for all dilutions of M-Traf3-/- #1-#3; S. epidermidis for all dilutions of M-Traf3-/- #2 and #3; E. faecalis for all dilutions of M-Traf3-/- #2 and #3; an intestinal commensal bacteria protein at ˜90 kDa of SI and colon from both LMC and M-Traf3-/- mice for all dilutions of M-Traf3-/- #3); bands of approximately the same molecular weight in different lanes (different bacterial strain's proteins) are denoted with a single “*” (red asterisk) on each blot.

FIG. 2 presents representative Western blots demonstrating that serum samples of B cell-specific Traf3-deficient (B-Traf3-/-) mice with spontaneous B lymphomas (B1 lymphomas or splenic marginal zone lymphomas (MZL)) contained high titers of antibodies specific for commensal bacterial proteins (the same as those detailed in the description of FIG. 1 above). Bacterial proteins were probed with serum samples at 1:500 (the left panel), 1:5000 (the middle panel), or 1:50000 (the right panel) dilutions. * (blue asterisk), mouse Ig light chain internal control (the same as FIG. 1); * (red asterisk), bacterial protein antigens specifically detected by serum Abs of B-Traf3-/- mice with B lymphomas (B1 lymphomas or MZL) (E. coli DH5α for all dilutions of B-Traf3-/- #2; K. pneumoniae for all dilutions of B-Traf3-/- #2; S. epidermidis for all dilutions of B-Traf3-/- #1; E. faecalis for all dilutions of B-Traf3-/- #1 and #3; intestinal commensal bacteria proteins of LMC colon for all dilutions of B-Traf3-/- #1-#3); bands of approximately the same molecular weight in different lanes (different bacterial strain's proteins) are denoted with a single “*” (red asterisk) on each blot.

FIG. 3 shows that plasma samples of human patients with non-Hodgkin lymphomas (NHL) contained high titers of antibodies specific for commensal bacterial proteins. Representative Western blots are shown (the same as those detailed in the description of FIG. 1 above). Bacterial proteins were probed with plasma samples at 1:500 (the left panel), 1:5000 (the middle panel), or 1:50000 (the right panel) dilutions. * (blue asterisk), a non-specific S. epidermidis protein antigen at ˜110 kDa directly recognized by the HRP-conjugated goat anti-human IgG (H+L) (2°) Ab is marked as an internal control (S. epidermidis for all dilutions of Healthy donor and Patients #1-#3); * (red asterisk), bacterial protein antigens specifically detected by plasma Abs of human patients with NHL (E. coli—DH5α for 1:500 and 1:5000 dilutions of Patient #1 and all dilutions of Patient #2; E. coli—Stbl3 for all dilutions of Patients #2 and #3; K. pneumoniae for all dilutions of Patient #1 and #2; E. faecalis for all dilutions of Patients #2 and #3; an intestinal commensal bacteria protein at ˜40 kDa of M-Traf3-/- SI and colon for all dilutions of Patient #3); bands of approximately the same molecular weight in different lanes (different bacterial strain's proteins) are denoted with a single “*” (red asterisk) on each blot.

FIGS. 4A-4D show that depletion of commensal bacteria by broad-spectrum antibiotic treatment prevented spontaneous B lymphoma development in aging myeloid cell-specific Traf3-deficient (M-Traf3-/-) mice. Male and female mice (8-10-month-old) were treated with 1 mg/ml ampicillin, 1.6 mg/ml sulfamethoxazole and 0.32 mg/ml trimethoprim in drinking water for 4 months to deplete commensal bacteria. FIGS. 4A-4C demonstrate that antibiotic treatment significantly prolonged the survival of M-Traf3-/- mice. P value between the two curves of each graph was determined by the Mantel-Cox log-rank test. FIG. 4D illustrates that antibiotic treatment abrogated spontaneous B lymphoma development in M-Traf3-/- mice.

FIGS. 5A-5D present that depletion of commensal bacteria by broad-spectrum antibiotic treatment inhibited spontaneous B lymphoma development in aging B cell-specific Traf3-deficient (B-Traf3-/-) mice. Male and female mice (2-3-month-old) were treated with 1 mg/ml ampicillin, 1.6 mg/ml sulfamethoxazole and 0.32 mg/ml trimethoprim in drinking water for 4 months to deplete commensal bacteria. FIGS. 5A-5C show that antibiotic treatment significantly improved the survival of B-Traf3-/- mice. P value between the two curves of each graph was determined by the Mantel-Cox log-rank test. FIG. 5D demonstrates that antibiotic treatment attenuated spontaneous B lymphoma development in B-Traf3-/- mice.

FIGS. 6A-6B show that serum samples of M-Traf3-/- and/or B-Traf3-/- mice with spontaneous B lymphomas contained high titers of antibodies specific for commensal bacterial antigens as determined by ELISA. In addition, FIG. 6B demonstrates that broad-spectrum antibiotic treatment markedly inhibited the serum titers of antibodies specific for commensal bacterial antigens in B-Traf3-/- mice. P values shown were analyzed by t test (FIG. 6A) or ANOVA (FIG. 6B).

FIGS. 7A-7B demonstrates that B-Traf3-/- mice with spontaneous B lymphomas contained antibody-producing cells that share the immunophenotypic characteristics with splenic B lymphoma cells and that sterile commensal bacterial antigens can induce the generation of antibody-producing cells from B-Traf3-/- splenic B lymphoma cells in ex vivo culture. FIG. 7A shows representative FACS profiles of splenocytes and BM cells showing CD138+ antibody-producing cells in the spleen and BM. Aging B-Traf3-/- mice with spontaneous B lymphomas contained CD138+ antibody-producing cells that share the immunophenotypic characteristics with splenic B lymphoma cells and were identified as B220+CD3−IgM^{hi}CD21−CD23−CD11b^{int}, which were not detected in aging LMC mice. FIG. 7B shows that sterile commensal bacterial antigens can also induce the generation of antibody-producing plasmablasts/plasma cells from B-Traf3-/- splenic B lymphoma cells in ex vivo culture. FIG. 7B presents FACS profiles of splenic B cells of aging LMC mice or young adult B-Traf3-/- mice and splenic B lymphoma cells of aging B-Traf3-/- mice after cultured ex vivo in the presence of 10 μg/ml of sterile soluble antigens of E. coli DH5α or 2 μg/ml agonistic anti-CD40 Abs plus 20 ng/ml IL-4, respectively, for 4 days.

FIGS. 8A-8D illustrate that sterile commensal bacterial antigens induce surface B cell receptor (BCR) internalization and Btk phosphorylation in splenic B lymphoma cells of B-Traf3-/- mice. IgM^{hi} splenic B lymphoma cells of 3 different individual B-Traf3-/- mice that contained high serum titers of anti-E. coli-specific Abs were used in the ex vivo culture experiments. Splenocytes of aging LMC mice, young adult B-Traf3-/- mice, or aging B-Traf3-/- mice with E. coli-specific B lymphomas were cultured in the absence (Media) or presence of 10 μg/ml of sterile soluble antigens of E. coli DH5α (EC) for 10 min (10′) or 30 min (30′). FIG. 8A presents representative FACS profiles showing the gating of splenic B cell subsets. Follicular (FO) B cells were gated as B220+CD23+CD21^{int}, marginal zone (MZ) B cells were gated as B220+CD23^{int}CD21+, and double negative (DN) B cells were gated as B220+CD23−CD21−. FIG. 8B shows an exemplary FACS histogram overlay comparing the staining intensity of surface IgM (BCR) and intracellular phosphorylated Btk (p-Btk). Graphical results of the geometric mean (GM) of surface IgM levels as determined by FACS analyses are presented in FIG. 8C. FIG. 8D illustrates graphical results showing the fold of change of the geometric mean (GM) of p-Btk levels in B lymphoma cells of B-Traf3-/- mice as determined by Phospho-Flow. P values between each treatment group and the Media control were analyzed by ANOVA: **, P<0.01.

FIG. 9 presents survival and proliferation data of human malignant B cells in culture as stimulated by sterile soluble commensal bacterial antigens. Human malignant B cell lines were cultured in the absence (Media) or presence of various concentrations (1:2 serial dilutions starting from 20 μg/ml) of sterile soluble commensal bacterial antigens for 72 h, and then viable cells of each condition were determined by MTT assay. The graphs depict the peak growth-stimulatory effects of each cell line by each commensal bacterial strain-derived soluble antigens as determined by MTT assay. P values between each treatment group and the Media control were analyzed by ANOVA: ns, P>0.05; *, P<0.05; **, P<0.01; ***, P<0.001.

FIGS. 10A-10B demonstrate that sterile commensal bacterial antigens induced the expression of chemokine receptors, adhesion molecules, and immunosuppressive ligands in human malignant B cells in culture. Human malignant B cell lines were cultured in the absence (Media) or presence of 10 μg/ml of sterile soluble antigens of E. coli DH5α (EC) or K. pneumoniae (KP) for 2 h, 24 h, or 72 h (FIG. 10A) or 72 h (FIG. 10B). FIG. 10A shows transcript expression levels of the chemokine receptor CCR7, the adhesion molecule CD44, and the immunosuppressive ligands PD-L1 (CD274) and PD-L2 (CD273) analyzed by quantitative real-time PCR. FIG. 10B demonstrates surface protein levels of the chemokine receptor CCR7, the adhesion molecule CD44, and the immunosuppressive ligand PD-L1 on Daudi cells as determined by FACS analysis. P values between each treatment group and the Media control were analyzed by ANOVA: *, P<0.05; **, P<0.01; ***, P<0.001.

FIG. 11 presents Western blots showing that sterile soluble commensal bacterial antigens engage BCR and TLR signaling events in cultured human malignant B cells. Daudi cells were cultured in the absence (Media) or presence of 10 μg/ml of sterile soluble commensal bacterial molecules (the same as those detailed in FIG. 10 above) for the indicated time periods. Blots shown are representative of 3 independent experiments.

### DETAILED DESCRIPTION

Detailed embodiments of the present disclosure are described herein; however, it is to be understood that the disclosed embodiments are merely illustrative of the disclosure that may be embodied in various forms. In addition, each of the examples given in connection with the various embodiments of the disclosure is intended to be illustrative, and not restrictive.

Gut microbiota has been suggested to be involved in the development of some cancers, including the development and progression of hematological malignancies. TNF receptor-associated factor 3 (TRAF3), a cytoplasmic adaptor protein, is a tumor suppressor in several human B-cell lineage neoplasms. Homozygous deletions and inactivating mutations of the human TRAF3 gene were detected in multiple myeloma (MM), Hodgkin lymphoma (HL), B-cell chronic lymphocytic leukemia (B-CLL), and non-Hodgkin lymphoma (NHL), including diffuse large B-cell lymphoma (DLBCL), splenic marginal zone lymphoma (MZL), mantle cell lymphoma (MCL), and Waldenström's macroglobulinemia. Myeloid cell-specific murine Traf3-deficient (M-Traf3-/-) and B cell-specific Traf3-deficient (B-Traf3-/-) mice models (see, e.g., Lalani et al., J. Immunol., 194:334-348, 2015; Moore et al., Leukemia 26:1122-1127, 2012, which is incorporated herein in its entirety for these mice model teachings) are used here and were shown to contain high titers of immunoglobulin antibodies specific for commensal bacterial antigens (e.g., proteins, glycoproteins, lipoproteins, carbohydrates, lipids, or nucleic acids). Non-Hodgkin lymphoma human plasma samples also contained high titers of immunoglobulin antibodies specific for commensal bacterial antigens.

Disclosed herein are embodiments directed to novel methods (e.g., Western blot and ELISA methods) for detecting commensal bacteria-specific antibodies, such as commensal bacteria-specific immunoglobulin antibodies (e.g., IgG (IgG1, IgG2, IgG3, IgG4); IgA (IgA1, IgA2); IgM; IgD; IgE), in a blood sample (e.g., plasma, serum) for early detection of B cell malignancies and for early detection of relapse or recurrence of a B cell malignancy in remission patients previously diagnosed as suffering from a B cell malignancy. Also, disclosed are embodiments directed to apparatuses for use with the novel methods described here and methods of treating B cell malignancies.

### Method of Detection in at Least Some Embodiments

In some embodiments, a method of detecting the presence of at least one commensal bacteria-specific antibody in a subject, where the subject is suspected of suffering from a B cell malignancy or a relapse or a recurrence of a B cell malignancy. For example, the disclosure comprises methods of early detection of B cell malignancies in a subject by exploiting commensal bacteria, which are good bacteria or beneficial microbes that secrete products to beneficially modulate the host and promote health. However, if there are issues, stresses, inflammation, infections, a temperature change, or when the host subject's immune system is compromised by genetic or environmental factors, commensal bacteria can become opportunistic, where the commensal bacteria, or antigens or ligands thereof, gain aberrant access to B cells and thus stimulate B cell malignancies or a recurrence of a B cell malignancy, or induce genetic mutations that cause cancer, such as B cell malignancies. One embodiment is directed to a method, comprising: (a) contacting at least one commensal bacterial antigen, such as a protein, a glycoprotein, a lipoprotein, a carbohydrate, a lipid, or a nucleic acid, and a sample from a subject suspected of or suffering from a B cell malignancy or a relapse or recurrence of a B cell malignancy, thereby forming a contacted sample. The sample from a subject can be a blood sample, such as a serum or plasma, from the subject. Additional biological samples from a subject for use in embodiments described here can also include ascites (i.e., fluid buildup in the abdomen), pleural fluid (fluid buildup in the lung), and tissue fluid or interstitial fluid (i.e., fluid from the spaces around cells). Advantageously, the method of at least one embodiment described here does not require the presence of any malignant B cells in the sample of a subject, which is distinct from all of the existing diagnostic approaches of B cell malignancies, and needs only a small volume of sample from the subject, for example, 5 microliters (μl) or greater (e.g., 7 μl, 9 μl, 11 μl, 13 μl, 15 μl, 17 μl, 19 μl, 21 μl, 23 μl, 25 μl, 50 μl, 75 μl, 125 μl, 175 μl, 225 μl, 275 μl, 325 μl, 375 μl, 425 μl, 475 μl, 525 μl, 575 μl, 625 μl, 675 μl, 725 μl, 775 μl, 825 μl, 875 μl, 925 μl, 975 μl, 1025 μl); 1 milliliter (ml) or less (e.g., 0.9 ml, 0.8 ml, 0.7 ml, 0.6 ml, 0.5 ml, 0.4 ml, 0.3 ml, 0.2 ml, 0.1 ml, 0.09 ml, 0.08 ml, 0.07 ml, 0.06 ml, 0.05 ml, 0.04 ml, 0.03 ml, 0.02 ml, 0.01 ml, 0.009 ml, 0.008 ml, 0.007 ml, 0.006 ml, 0.005 ml); 5 μl to 1 ml (e.g., 6 μl-950 μl; 7 μl-850 μl; 8 μl-750 μl; 9 μl-650 μl; 10 μl-550 μl; 11 μl-450 μl; 12 μl-350 μl; 13 μl-250 μl; 14 μl-150 μl; 15 μl-50 μl; 16 μl-48 μl; 17 μl-46 μl; 18 μl-44 μl; 19 μl-42 μl; 20 μl-40 μl; 21 μl-38 μl; 22 μl-36 μl; 23 μl-34 μl; 24 μl-32 μl; 25 μl-30 μl), where the sample can be a blood sample (e.g., serum, plasma), an ascites sample, a tissue fluid or interstitial fluid sample, or any other contemplated sample that contains at least one commensal bacteria-specific antibody from a subject. The method described here, in some embodiments, can further comprise (b) incubating the contacted sample and a detection antibody. In some embodiments, the detection antibody comprises at least one of: specificity for at least one commensal bacteria-specific antibody; specificity for at least one immunoglobulin light chain (κ or λ) of the subject (e.g., human; canine; feline), where at least one commensal bacteria-specific antibody comprises at least one immunoglobulin light chain (κ or λ); or specificity for at least one immunoglobulin heavy chain of the subject selected from the group consisting of: IgG (e.g., IgG1, IgG2, IgG3, IgG4), IgA (e.g., IgA1, IgA2), IgM, IgD, IgE, and any combination thereof, where the at least one commensal bacteria-specific antibody comprises at least one heavy chain. One embodiment of the method can comprises a blood sample from a subject suspected of suffering from a B cell malignancy or a relapse or recurrence of a B cell malignancy, where the blood sample comprises at least one immunoglobulin light chain of an antibody (κ or λ), where the antibody is selected from IgG1, IgG2, IgG3, IgG4, IgA1, IgA2, IgM, IgD, IgE, or any combination thereof, and the antibody is specific for at least one commensal bacterial antigen, such as at least one attached to an apparatus comprising at least one commensal bacterial antigen. Non-limiting examples of the apparatus include a membrane, a multi-well plate, a bead, a chip, or a slide. For example, the apparatus is selected from the group consisting of: a nitrocellulose membrane; a polyvinylidene fluoride (PVDF) membrane; a polystyrene multi-well plate; a polyvinyl chloride multi-well plate; a bead; a chip (e.g., a microfluidic chip or microchip); and a slide, where the apparatus is configured to enable or allow commensal bacterial antigen coating or protein deposition or conjugation. In some embodiments, the method described here can further comprise reducing background interference on the apparatus. For example, reducing background interference can include blocking the apparatus comprising the at least one commensal bacterial antigen before (a) contacting and/or incubating the at least one commensal bacterial antigen and blood sample from the subject suspected of or suffering from a B cell malignancy or a relapse or a recurrence of a B cell malignancy, where blocking the apparatus can reduce or prevent the subject's antibodies from non-specifically binding to the apparatus. Another example of reducing background interference comprises washing the apparatus comprising the contacted sample before (b) incubating the contacted sample and the detection secondary (2°) antibody, after (b) incubating the contacted sample and the detection antibody, or both before and after (b) incubating the contacted sample and the detection antibody, in order to remove unbound antibodies and to reduce or prevent antibodies from non-specifically binding to the apparatus. The detection antibody comprises a detectable label, such as but not limited to, a detectable label selected from: an enzyme label, a fluorescent label, or a biotin label. Non-limiting examples of detectable labels include: horseradish peroxidase (HRP), alkaline phosphatase (AP), β-galactosidase, glucose oxidase, glucose-6-phosphate dehydrogenase, β-N-acetylglucosaminidase, invertase, xanthine oxidase, biotin, Fluorescein Isothiocyanate (FITC), Tetramethylrhodamine-5-(and 6)-isothiocyanate (TRITC), Alexa Fluor® (AF) dye (e.g., AF 350, AF 405, AF 430, AF 488, AF 500, AF 514, AF 532, AF 546, AF 555, AF 568, AF 594, AF 610, AF 633, AF 635, AF 647, AF 660, AF 680, AF 700, AF 750, AF 790), Allophycocyanin (APC), phycoerythrin (PE), Brilliant violet (BV) dye (e.g., BV 421, BV 480, BV 510, BV 570, BV 605, BV 650, BV 711, BV 750, BV 785), Peridinin-chlorophyll-protein (PerCP), Pacific blue, Pacific orange, PE/Dazzle 594, APC Fire 750, Super Bright 436, Zombie Green, Texas Red, Rhodamine, and cyanine (Cy) dye (e.g., Cy2, Cy3, Cy5). In some embodiments, the method further comprises (c) detecting the detection antibody, where the detection antibody reflects the presence of at least one commensal bacteria-specific antibody in the subject. For example, the detection occurs by an immunoassay. Non-limiting immunoassays include Western blot, enzyme-linked immunosorbent assay (ELISA), flow cytometric immunoassay (e.g., fluorescence-activated cell sorting (FACS)), enzyme immunoassay (EIA), fluoroimmunoassay (FIA), chemiluminescent immunoassay (CLIA), counting immunoassay (CIA), and radioimmunoassay (RIA). A person of ordinary skill in the art understands an appropriate detection antibody for use with the immunoassay of choice. In other embodiments, the method described here comprises (c) detecting the detection antibody in the blood sample from a subject in remission and suspected of suffering from a relapse or a recurrence of a B cell malignancy, where the detection antibody reflects the presence of at least one commensal bacteria-specific antibody and/or at least one commensal bacterial antigen that gains access to B cells in the subject. One embodiment provides the described method of (c) detecting the detection antibody in the blood sample of a subject, where the blood sample from the subject suspected of or suffering from a B cell malignancy contains at least one commensal bacteria-specific antibody comprising a titer of equal to or more than 10-fold (e.g., 10 to 10⁵; 10-100,000; 100-10,000; 1,000-5,000) as compared to the titer of a healthy subject, where the healthy subject does not suffer from a B cell malignancy or an aberrant B cell response to commensal bacterial antigen(s) indicative of a B cell malignancy, and as a result indicates that the subject suffers from a B cell malignancy. Another embodiment provides the described method of (c) detecting the detection antibody in the blood sample of a subject, where the blood sample from the subject contains at least one commensal bacteria-specific antibody comprising a titer of equal to or more than 5-fold (e.g., 5 to 10⁵; 5-100,000; 50-50,000; 100-10,000; 500-5,000) as compared to a titer baseline of the subject in remission or diagnosed in remission of a B cell malignancy, and as a result indicates that the subject has relapsed or a recurrence of the B cell malignancy. Non-limiting examples of a B cell malignancy include: diffuse large B-cell lymphoma (DLBCL), follicular lymphoma (FL), Burkitt lymphoma (BL), mantle cell lymphoma (MCL), splenic marginal zone lymphoma (MZL), chronic lymphocytic leukemia (CLL), mucosa-associated lymphoid tissue lymphoma (MALT), multiple myeloma (MM), Waldenström macroglobulinemia (WM), hairy cell leukemia (HCL), and Hodgkin lymphoma (HL) as well as the precursor diseases of B cell malignancies such as monoclonal gammopathy of unknown significance (MGUS) and monoclonal B-cell lymphocytosis (MBL). Some embodiments provide the method described here further comprising treating the subject containing at least one commensal bacteria-specific antibody and/or one strain of commensal bacteria in an amount sufficient to be diagnosed with a B cell malignancy or diagnosed with a relapse or a recurrence of a B cell malignancy after having been in remission.

### Stages for B Cell Lymphoma diagnosis and for Relapse vs Newly Diagnosed Patients

In some embodiments, newly diagnosed subjects have a commensal bacteria-specific antibody titer that is more than or equal to (≥) 10-fold (in the range of 10 to 10⁵) as compared to healthy control subjects, which would be considered malignant. The higher the titer, the more severe the malignancy. See, TABLE 1 (showing an example of a potential use in the diagnosis of new patients with B cell malignancies).

Other embodiments of the disclosure are directed to early detection of relapse or recurrence of a B cell malignancy in a subject, where the commensal bacteria-specific antibody titers are more than or equal to (≥) 5-fold as compared to a baseline titer count of the same subject after complete remission. See, TABLE 2 (showing an example of a potential use in early detection of relapse or recurrence of B cell malignancies in patients after treatment and complete remission). Accordingly, testing titer count predicts the risk of relapse or recurrence.

TABLE 1
Newly Diagnosed Subjects' Titer Fold
As Compared To Healthy Control Subjects

| Titer Fold of healthy | |
|---|---|
| control subjects | Stage of B cell lymphoma |
| 10-100 | Stage 1 or Stage 2 (early-stage) |
| 100-100,000 | Stage 3 or Stage 4 (advanced-stage) |

TABLE 2
Relapsed Subjects' Titer Fold As Compared
to Same Subject's Remission Baseline

| Titers Fold of Same Subject's | |
|---|---|
| Baseline after Complete Remission | Stage of B cell lymphoma |
| 5-10 | Risk of Relapse or Recurrence |
| 10-100 | Early-stage of Relapse |
| 100-100,000 | Advanced-stage of Relapse |

In one embodiment, a method of the disclosure comprises: a method, comprising: (a) contacting a plurality of commensal bacterial antigens (e.g., proteins; glycoproteins; lipoproteins; carbohydrates; lipids; nucleic acids) and a sample (e.g., blood) from a subject suspected of or suffering from a B cell malignancy or a relapse or recurrence of a B cell malignancy, thereby forming a contacted sample on an apparatus; (b) incubating the contacted sample and a detection enzyme-conjugated antibody (that binds to at least one immunoglobulin light chain (κ or λ) of the subject species (e.g., human; canine; feline); at least one immunoglobulin heavy chain of the subject species (e.g., human; canine; feline) (e.g., IgG (IgG1, IgG2, IgG3, IgG4); IgA (IgA1, IgA2); IgM; IgD; IgE)); (c) detecting the detection enzyme-conjugated antibody, wherein the detection enzyme-conjugated antibody reflects the presence of a plurality of commensal bacteria-specific antibodies in the subject; and (d) treating the subject containing the plurality of commensal bacteria-specific antibodies. The method of the disclosure further comprising attaching the plurality of commensal bacterial proteins or antigens to the apparatus or obtaining an apparatus comprising the plurality of commensal bacterial proteins or antigens prior to contacting the sample from the subject suspected of or suffering from a B cell malignancy or a relapse or recurrence of a B cell malignancy to the plurality of commensal bacterial proteins or antigens attached to the apparatus. Additional methods of the disclosure further comprise reducing background interference on the apparatus. For example, reducing background interference can include blocking the apparatus comprising the at least one or a plurality of commensal bacterial protein(s) or antigen(s) before (a) contacting and/or incubating the at least one commensal bacterial antigen or protein and blood sample from the subject suspected of or suffering from a B cell malignancy or a relapse or a recurrence of a B cell malignancy, where blocking the apparatus can reduce or prevent antibodies of the subject from non-specifically binding to the apparatus. Another example of reducing background interference comprises washing the apparatus comprising the contacted sample before (b) incubating the contacted sample and the detection secondary (2°) antibody, after (b) incubating the contacted sample and the detection antibody, or both before and after (b) incubating the contacted sample and the detection antibody, in order to remove unbound antibodies and to reduce or prevent antibodies from non-specifically binding to the apparatus.

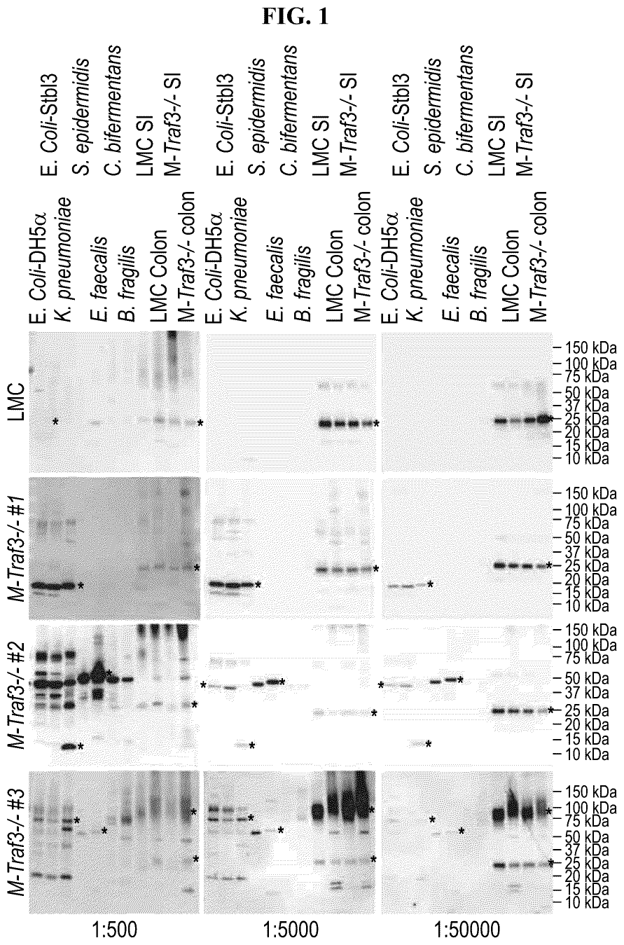

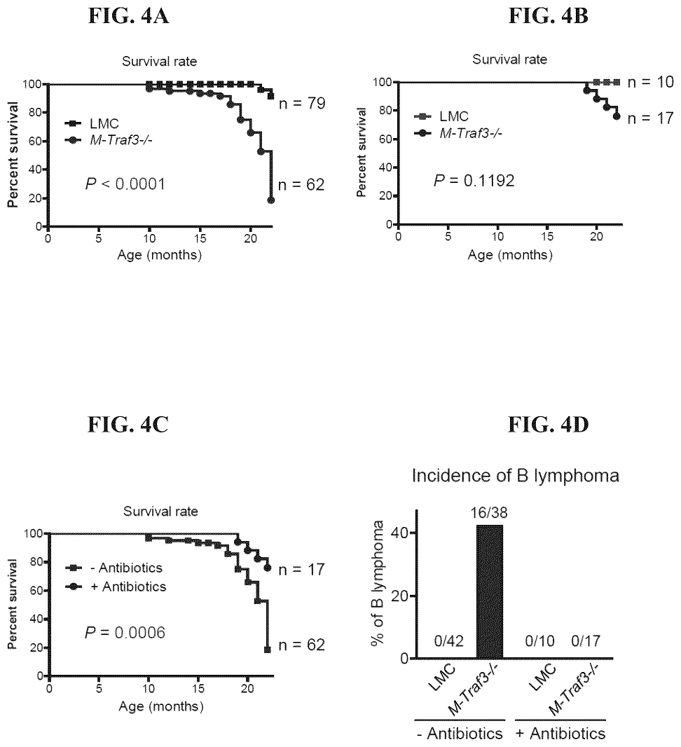

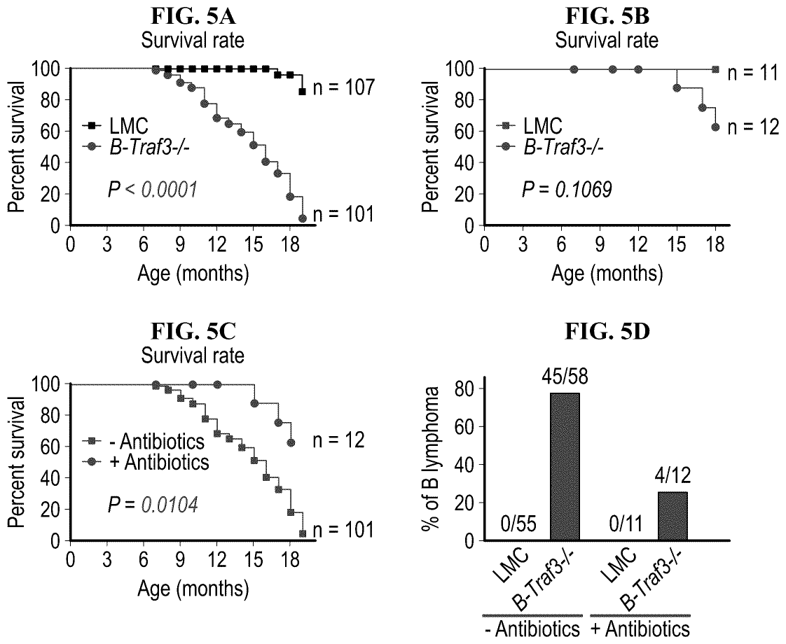

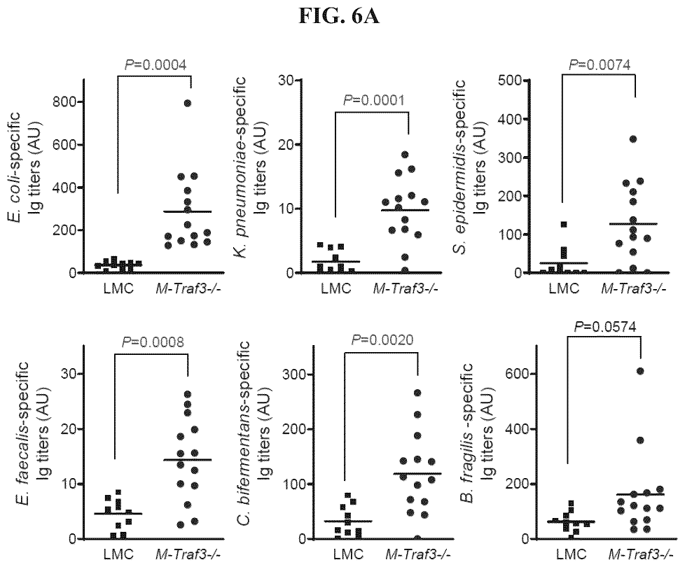

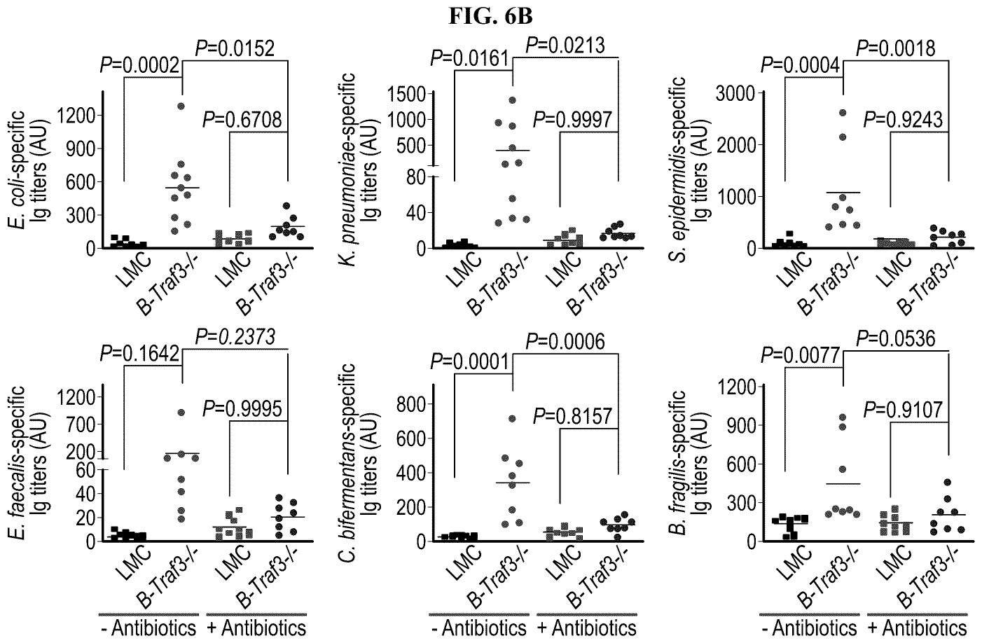

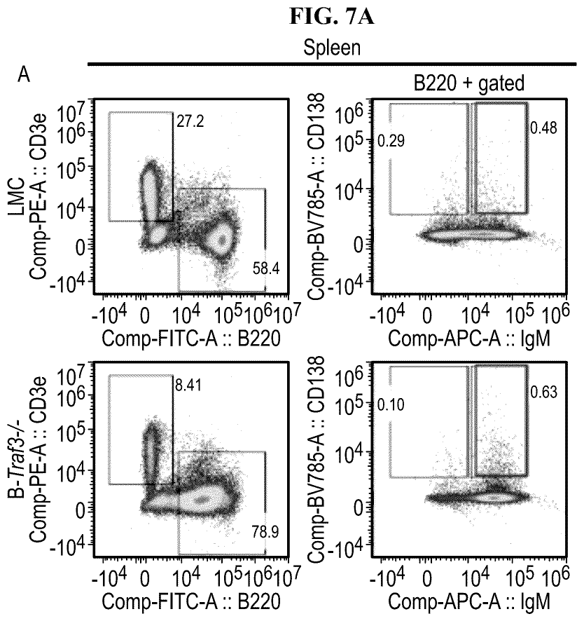

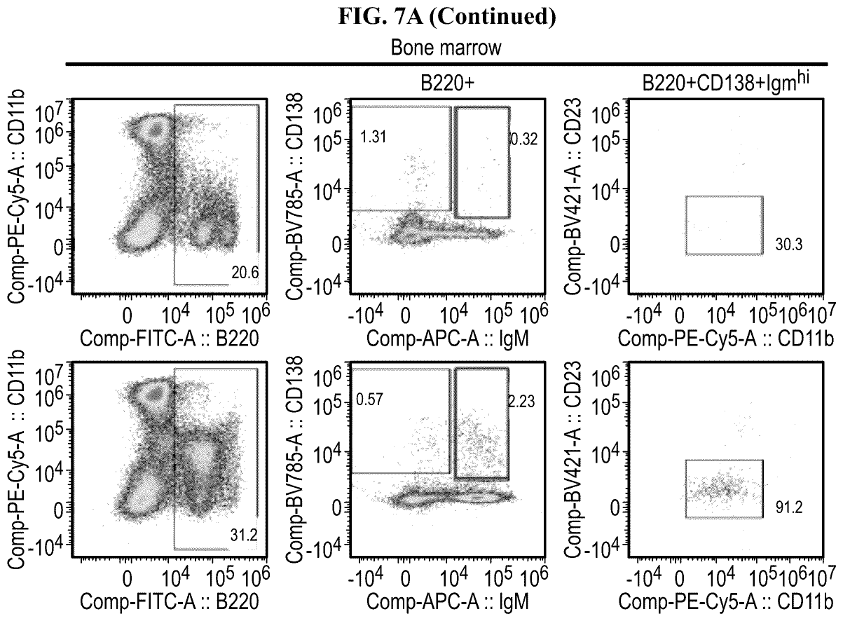

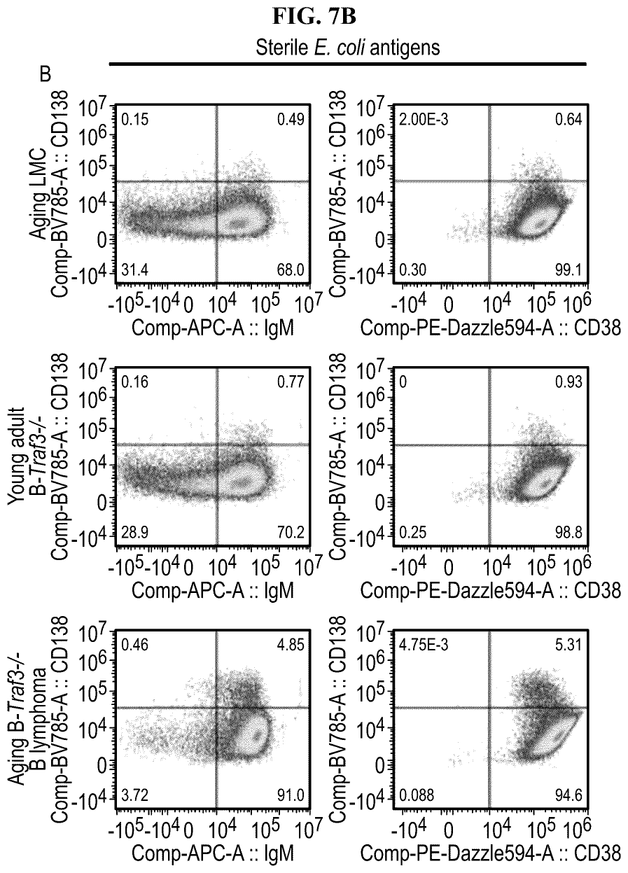

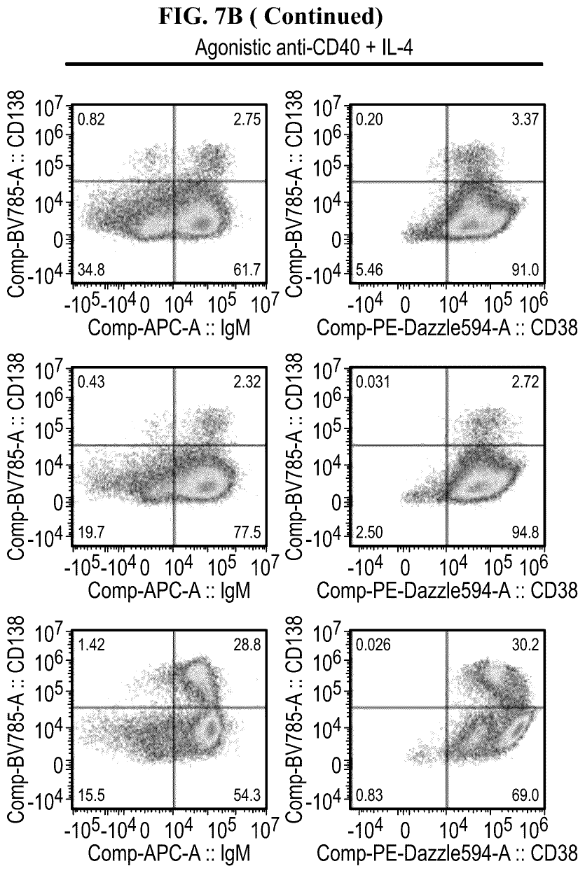

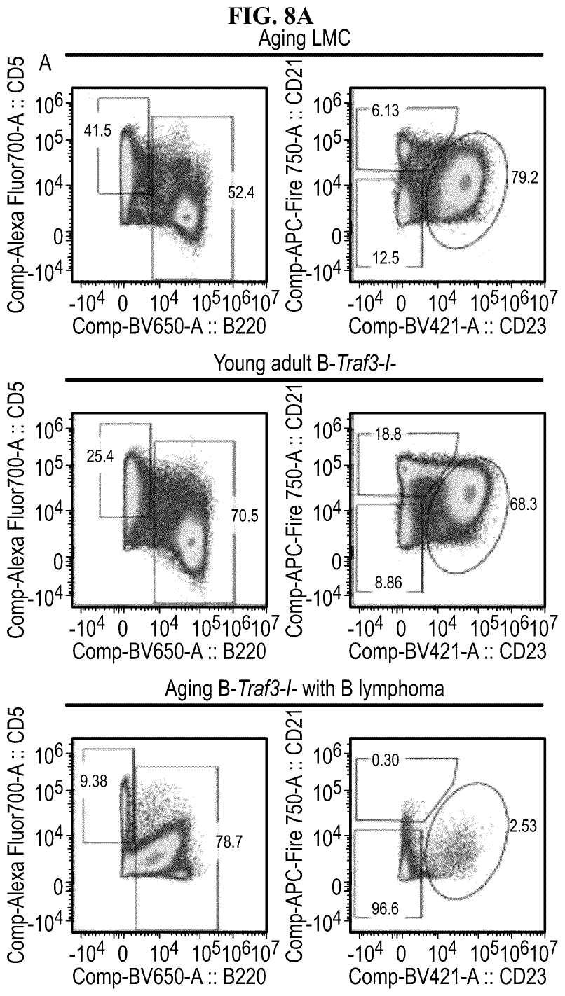

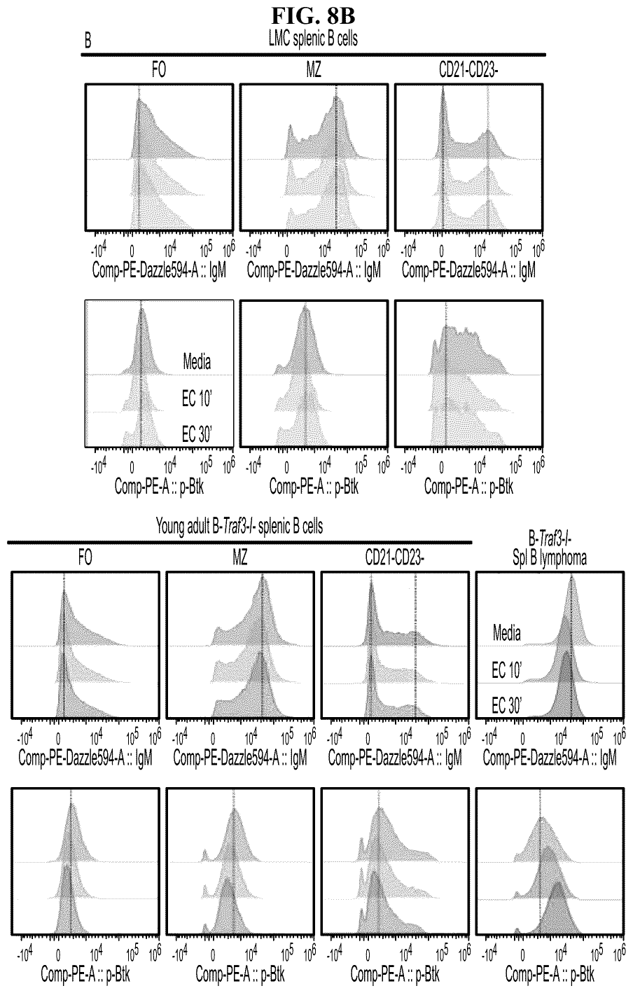

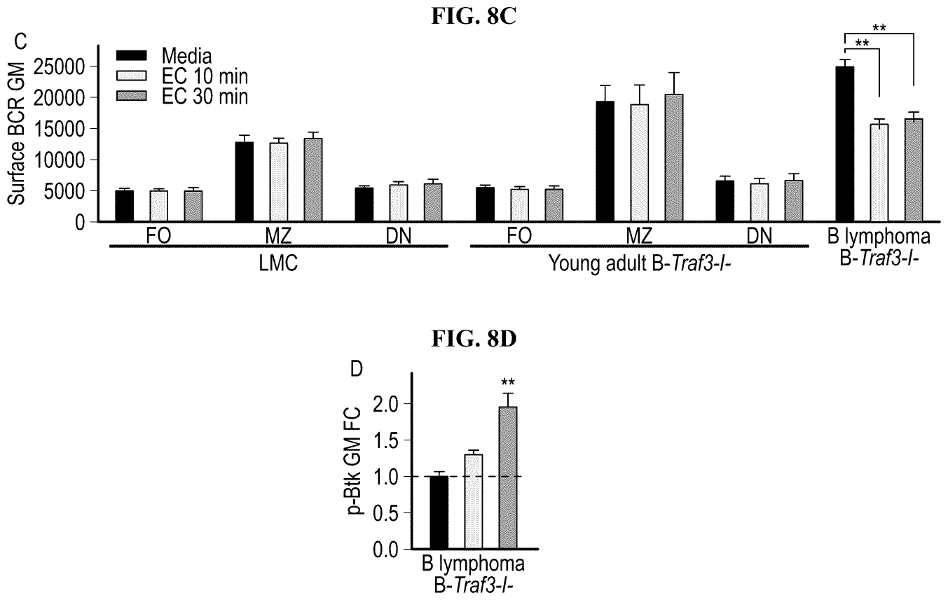

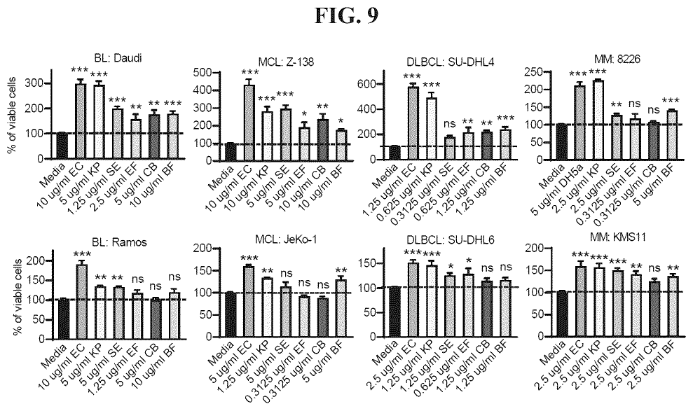

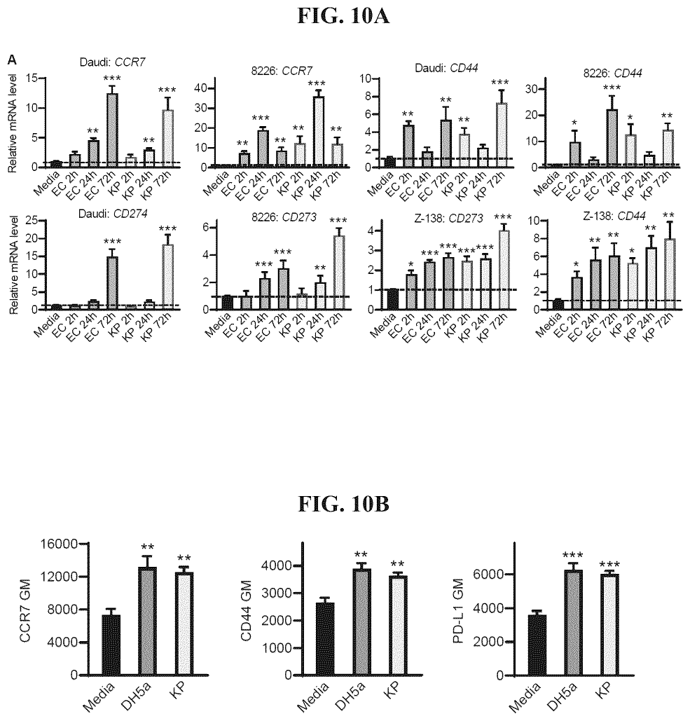

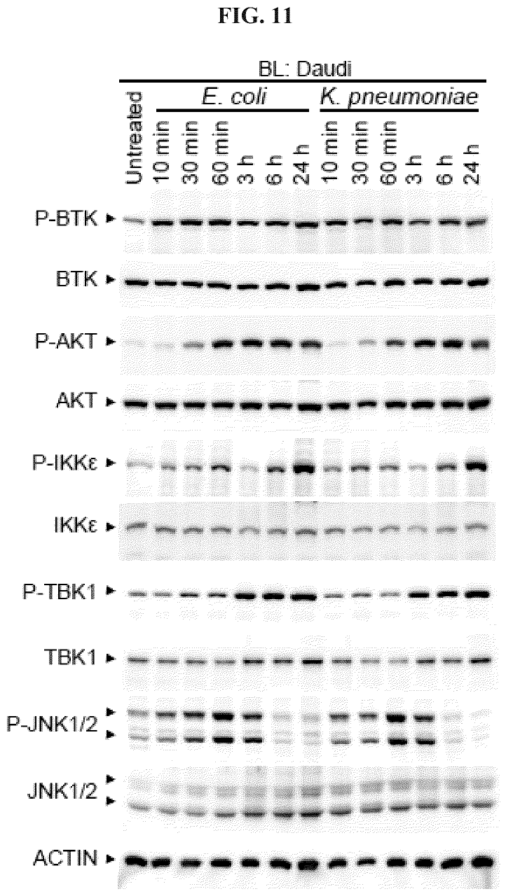

The Western blots of FIGS. 1, 2, and 3 show that blood (serum or plasma) samples of both mice and human subjects with B cell lymphomas contain high titers of antibodies against commensal bacterial antigens in view of detection at 1:5000 and 1:50000 dilutions. FIGS. 4 and 5 demonstrate that antigens and/or ligands derived from commensal bacteria are a major driver of B lymphoma development and relapse, as depletion of commensal bacteria by broad-spectrum antibiotic treatment prevented or inhibited B lymphoma development in mouse models with spontaneous B lymphomas. The ELISA data of FIGS. 6A-6B further verify that serum samples of mice with B lymphomas contain high titers of antibodies specific for commensal bacterial antigens, which could be reduced by broad-spectrum antibiotic treatment. The FACS data of FIGS. 7A-7B demonstrate that B-Traf3-/- mice with spontaneous B lymphomas contained antibody-producing cells that share the immunophenotypic characteristics with splenic B lymphoma cells in the bone marrow (BM). The data also show that sterile commensal bacterial antigens can induce the generation of CD138+ antibody-producing cells from splenic B lymphoma cells of B-Traf3-/- mice in ex vivo culture. The FACS data of FIG. 8 illustrate that sterile commensal bacterial antigens induced surface B cell receptor (BCR) internalization and Btk phosphorylation in splenic B lymphoma cells of B-Traf3-/- mice, indicating that the BCRs expressed on malignant B cells can recognize and react to commensal bacterial antigens. The MTT assay data of FIG. 9 demonstrate that sterile soluble commensal bacterial molecules can stimulate the survival and proliferation of human malignant B cell lines in culture. The gene expression data of FIG. 10 shows that sterile soluble commensal bacterial molecules can induce the expression of chemokine receptors, adhesion molecules, and immunosuppressive ligands in human malignant B cells in culture. The Western blot data of FIG. 11 demonstrate that sterile soluble commensal bacterial molecules can engage BCR and TLR signaling events in cultured human malignant B cells.

### Methods of Treatment

Based on the findings that treatment of mouse models of B cell malignancies (DLBCL, FL, MZL, and B1 lymphomas) with antibiotics depleted commensal bacteria, thereby preventing or substantially inhibiting B cell lymphoma development and progression (see, e.g., FIGS. 4, 5, 6B), additional embodiments of the disclosure further comprise treating methods for B cell malignancies that can be linked to the specific detection methods or used independently. Some embodiments of the disclosure are directed to methods of detecting at least one commensal bacteria-specific antibody and/or at least one strain of commensal bacteria in a sample from a subject suspected of or suffering from a B cell malignancy or a relapse or recurrence of a B cell malignancy, further comprising treating the subject comprising at least one commensal bacteria-specific antibody and/or at least one strain of commensal bacteria, wherein the subject comprises an amount sufficient to be diagnosed with a B cell malignancy or the subject in remission is diagnosed with a relapse or a recurrence of a B cell malignancy. The methods described here provide for sensitive and early detection of a B cell malignancy in a subject or in a subject in remission previously diagnosed with a B cell malignancy.

The methods of treating a subject diagnosed with a B cell malignancy described here comprise: (i) administering to the subject, at least one antibiotic (selected according to the subject's antibiotic tolerance and resistance profile) in an effective amount sufficient to: reduce at least one strain of commensal bacteria, alleviate a symptom of the B cell malignancy, palliate the B cell malignancy, slow the progression of the B cell malignancy, induce remission, or combinations thereof; (ii) monitoring a titer of commensal bacteria-specific antibodies in the subject; (iii) repeating the method of (a)-(c) or (a)-(d) (i.e., (a) contacting at least one commensal bacterial antigen (e.g., protein) and blood from a subject suspected of or suffering from a B cell malignancy or a relapse or recurrence of a B cell malignancy, thereby forming a contacted sample; (b) incubating the contacted sample and a detection antibody (that binds to at least one immunoglobulin light chain (κ or λ) of the subject species (e.g., human; canine; feline) or at least one immunoglobulin heavy chain (e.g., IgG (IgG1, IgG2, IgG3, IgG4); IgA (IgA1, IgA2); IgM; IgD; IgE) of the subject species; (c) detecting the detection antibody; and (d) treating the subject comprising at least one commensal bacteria-specific antibody); or (iv) combinations of (i)-(iii) thereof. Additional embodiments of methods of treatment of the disclosure comprise decreasing the commensal bacterial antigens or commensal bacteria in a subject suspected of or suffering from a B cell malignancy or a relapse or recurrence of a B cell malignancy. In some embodiments, the method comprises administering at least one antibiotic (according to the subject's antibiotic tolerance and resistance profile) by oral administration, intravenous administration, intramuscular administration, or combinations thereof. Additional embodiments provide for a treatment cycle comprising administering (e.g., orally, intravenously) at least one antibiotic (e.g., 2, 3, 4, 5) for one or more times per day (e.g., 2, 3, 4, 5, 6). In further embodiments, the treatment cycle comprises administering for at least 7 days consecutively or alternating days ((e.g., 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 50, 60, 70, 80, 90, 100, 110, 120, 130); 123 consecutive days or fewer (e.g., 122, 121, 120, 119, 118, 117, 116, 115, 114, 113, 112, 111, 110, 109, 108, 107, 106, 105, 104, 103, 102, 101, 100, 99, 98, 97, 96, 95, 94, 93, 92, 91, 90); or 7-130 consecutive days (e.g., 8-129; 9-128; 10-127; 11-126; 12-125; 13-124; 14-123; 15-122; 16-121; 17-120; 18-119; 19-118; 20-117; 21-116; 22-115; 23-114; 24-113; 25-112; 26-111; 27-110; 28-109; 29-108; 30-107; 31-106)). The methods of treating comprise administering at least one antibiotic, for example, selected (according to the subject's antibiotic tolerance and resistance profile) from the group consisting of: penicillin class, sulfonamide class, nitroimidazole class, glycopeptide class, quinolone class, lincosamide class, beta-lactam class, aminopyrimidine class, and combinations thereof. Further examples of the methods of treating described here include at least one antibiotic in a treatment regimen comprising ampicillin, sulfamethoxazole, trimethoprim, metronidazole, vancomycin, ciprofloxacin, levofloxacin, clindamycin, augmentin (i.e., amoxicillin/clavulanate), penicillin, cephalosporins, or combinations thereof. Other exemplary antibiotics include those for treating a urinary tract or bladder infection and/or ear infection, such as but not limited to, amoxicillin; amoxicillin/potassium clavulanate; streptomycin; gentamicin; neomycin/polymyxcin b/hydrocortisone; colistin/neomycin/thonzonium/hydrocortisone; nitrofurantoin; cephalexin; ciprofloxacin/dexamethasone; ciprofloxacin/hydrocortisone; ciprofloxacin; ofloxacin; ceftriaxone; fosfomycin; kanamycin; levofloxacin; imipenem/cilastatin; cefoxitin; and the like. Some embodiments of the disclosure provide methods of treating, where the at least one antibiotic in a treatment regimen disclosed here comprises, consists essentially of, or consists of at least two antibiotics (e.g., 3, 4, 5, 6), where the at least two antibiotics can be administered together, separately, simultaneously or essentially simultaneously, sequentially or essentially sequentially, or any combinations thereof. For example, the at least two antibiotics can comprise, consist essentially of, or consist of three antibiotics, wherein the three antibiotics can comprise, consist essentially of, or consist of ampicillin, sulfamethoxazole, and trimethoprim. Other embodiments are directed to the methods of the disclosure, where administering the at least one antibiotic in a treatment regimen disclosed here comprises administering, for example, ampicillin in an amount of 50 mg-1000 mg (e.g., 500 mg); and sulfamethoxazole and trimethoprim in an amount ranging from 2.5 mg/kg-20 mg/kg (e.g., 8 mg/kg-10 mg/kg), where sulfamethoxazole and trimethoprim are in a ratio of 5:1 (e.g., 50 mg sulfamethoxazole and 10 mg trimethoprim; 100 mg sulfamethoxazole and 20 mg trimethoprim; 200 mg sulfamethoxazole and 40 mg trimethoprim; 400 mg sulfamethoxazole and 80 mg trimethoprim; 800 mg sulfamethoxazole and 160 mg trimethoprim). For example, the at least one antibiotic in a treatment regimen disclosed in various embodiments here can be administered in an amount sufficient to treat the B cell malignancy, reduce the at least one strain of commensal bacteria, deplete at least one strain of commensal bacteria, or combinations thereof, where the amount of ampicillin comprises 50 mg or greater (e.g., 60, 70, 80, 90, 100, 200, 300, 400, 500, 600, 700, 800, 900, 1000, 1200, 1300, 1400, 1500); 1000 mg or less (e.g., 950, 850, 750, 650, 550, 450, 350, 250, 150, 50, 25); 50 mg-1000 mg (e.g., 65 mg-975 mg; 75 mg-875 mg; 85 mg-775 mg; 95 mg-675 mg; 105 mg-575 mg; 115 mg-475 mg; 125 mg-375 mg; 135 mg-275 mg; 145 mg-175 mg); the amount of sulfamethoxazole and trimethoprim comprises 2.5 mg/kg or greater (e.g., 3.5, 4.5, 5.5, 6.5, 7.5, 8.5, 9.5, 10.5, 11.5, 12.5, 13.5, 14.5, 15.5, 16.5, 17.5, 18.5, 19.5, 20.5); 20 mg/kg or less (e.g., 19, 18, 17, 16, 15, 14, 13, 12, 11, 10, 9, 8, 7, 6, 5, 4, 3, 2); or 2.5 mg/kg-20 mg/kg (e.g., 3.25 mg/kg-20.75 mg/kg; 4.25 mg/kg-19.75 mg/kg; 5.25 mg/kg-18.75 mg/kg; 6.25 mg/kg-17.75 mg/kg; 7.25 mg/kg-16.75 mg/kg; 8.25 mg/kg-15.75 mg/kg; 9.25 mg/kg-14.75 mg/kg; 10.25 mg/kg-13.75 mg/kg; 11.25 mg/kg-12.75 mg/kg), where sulfamethoxazole and trimethoprim are in a ratio of 5:1 (e.g., 50 mg sulfamethoxazole and 10 mg trimethoprim; 100 mg sulfamethoxazole and 20 mg trimethoprim; 200 mg sulfamethoxazole and 40 mg trimethoprim; 400 mg sulfamethoxazole and 80 mg trimethoprim; 800 mg sulfamethoxazole and 160 mg trimethoprim). Some embodiments of the methods of treating described here comprises administering ampicillin 1 time, 2 times, 3 times, or 4 times per day; administering ampicillin once every 24 hours, 12 hours, 8 hours, or 6 hours; or administering ampicillin 4 times per day at once every 6 hours. In additional embodiments of the methods of treating described here comprises administering sulfamethoxazole and trimethoprim 1 time, 2 times, 3 times, or 4 times per day; administering sulfamethoxazole and trimethoprim once every 24 hours, 12 hours, 8 hours, or 6 hours; or administering sulfamethoxazole and trimethoprim (in a ratio of 5:1) two times per day at once every 12 hours. For example, ampicillin, sulfamethoxazole, and trimethoprim are administered simultaneously or essentially simultaneously; sequentially or essentially sequentially; together or essentially together; or separately or essentially separately. In some embodiments, the methods of treating disclosed here comprise administering sulfamethoxazole and trimethoprim together. Additional embodiments of the disclosure comprise methods of treating the subject diagnosed with a B cell malignancy by administering at least one antibiotic in an effective amount sufficient to: reduce the at least one strain of commensal bacteria, alleviate a symptom of the B cell malignancy, palliate the B cell malignancy, slow the progression of the B cell malignancy, induce remission, or combinations thereof; and further comprising subjecting the subject to an additional standard of care treatment selected from the group consisting of: chemotherapy, radiation therapy, immunotherapy, stem cell transplant, targeted drugs, surgery, and combinations thereof. The methods of detecting and treating described here comprise a subject that is an animal or a mammal, selected from, for example, a human, a canine, a feline, a leporine, an equine, a bovine, a murine, and the like, or any mammal suspected of or suffering from a B cell malignancy or a relapse or recurrence of a B cell malignancy.

Further embodiments, incorporating various elements described here, such as but not limited to, the route of administration, the antibiotics, the effective amounts, the dosage regimen, and the like, provide methods of preventing, inhibiting, or decreasing development and/or progression of B cell malignancies in a subject diagnosed with such a B cell malignancy using the methods described here. The methods of preventing, inhibiting, or decreasing development and/or progression of B cell malignancies in the subject, comprising administering at least one antibiotic in an amount effective to kill, deplete, and/or slow the growth of commensal bacteria, thereby treating the subject suffering from a B cell malignancy. In additional embodiments, these methods can be used in combination with or without the methods of detecting described here.

### Apparatus

In some embodiments, the methods of the disclosure comprise an apparatus or the use of an apparatus. One embodiment provides for the apparatus described here, where the apparatus can be selected from: a membrane or a multi-well plate, where the membrane comprises a nitrocellulose membrane or a polyvinylidene fluoride (PVDF) membrane, or the multi-well plate comprises a polystyrene multi-well plate or polyvinyl chloride multi-well plate, or a bead or a chip (e.g., a microfluidic chip or microchip) or a slide, where the apparatus is configured to enable or allow commensal bacterial antigen coating or protein deposition or conjugation. In embodiments of the disclosure, the apparatuses described here comprise at least one commensal bacterial antigen, such as protein, or a plurality of commensal bacterial antigens, where representative opportunistic commensal bacteria (termed pathobionts) include, but not limited to, Escherichia coli (E. coli) (e.g., strain DH5α, Stbl3), Klebsiella pneumoniae (K. pneumoniae), Staphylococcus epidermidis (S. epidermidis), Enterococcus faecalis (E. faecalis), Clostridium bifermentans (C. bifermentans), Bacteroides fragilis (B. fragilis), and the like. Additional exemplary representative, but non-limiting commensal bacteria and opportunistic bacteria are provided here. See also, Chow et al. Curr Opin Immunol. 23: 473-480, 2011; Packey and Sartor. Curr Opin Infect Dis. 22: 292-301, 2009; TABLE 3 (Yang et al. Front Microbiol. 11:2029, 2020), TABLE 4 (Lagkouvardos et al. Nat Microbiol. 1:16131, 2016), TABLE 5 (Yang et al. Front Microbiol. 11:2029, 2020), and TABLE 6 (Chandra et al. Gut Microbes. 13:1979882, 2021).

TABLE 3
List of Representative Human Gut Commensal Bacteria

| Bacterial Species Name | Group Annotation |
|---|---|
| Bacteroides vulgatus | |
| Faecalibacterium prausnitzii | |
| Parabacteroides merdae | |
| Eubacterium rectale | |
| Escherichia coli* | Escherichia albertii/Escherichia |
| | fergusonii/Escherichia coli |
| Roseburia faecis | |
| Phascolarctobacterium faecium | |
| Bacteroides ovatus | |
| Alistipes shahii | |
| Roseburia intestinalis | |
| Parasutterella excrementihominis | |
| Eubacterium eligens | |
| Ruminococcus bromii | |
| Lactobacillus rogosae* | Lactobacillus rogosae/ |
| | Bacteroides galacturonicus |
| Prevotella copri | |
| Anaerostipes hadrus | |
| Bacteroides massilliensis | |
| Bacteroides dorei | |
| Ruminococcus faecis | |
| Sphingomonas echinoides | |
| Bacteroides stercoris | |
| Dorea formicigenerans | |
| Ochrobactrum cytisi* | Ochrobactrum cytisi/Ochrobactrum |
| | lupini/Ochrobactrum |
| | anthropi/Ochrobactrum tritici |
| Fusicatenibacter saccharivorans | |
| Sutterella wadswothensis | |
| Sphingomonas melonis | Sphingomonas melonis/Sphingomonas aquatilis |
| Bacteroides thetaiotaomicron | |
| Bacteroides coprophilus | |
| Blautia obeum | |
| Eubacterium hallii | |
| Roseburia inulinivorans | |
| Holdemanella biformis | |
| Bacteroides cellulosilyticus | |
| Eubacterium siraeum | |
| Coprobacter fastidiosus | |
| Intestinibacter bartlettii | |
| Subdoligranulum variabile | |
| Ruminococcus callidus | |
| Tsukamurella spongiae | |
| Blautia wexlerae | |
| Phascolarctobacterium succinatutens | |
| Ruminococcus lactaris | |
| Bacteroides clarus | |
| Blautia stercoris | |
| Eubacterium hadrum* | Eubacterium hadrum/Anareostipes hadrus |
| Butyrivibrio crossotus | |
| Coprobacter secundus | |
| Alistipes indistinctus | |
| Romboutsia ilealis | |
| Holdemania filiformis | |
| Catenibacterium mitsuokai | |
| Serratia quinivorans* | Serratia quinivorans/Serratia |
| | proteamaculans/Serratia grimesi |
| Parabacteroides johnsonii | |
| Enterobacter muelleri | |
| Anaerotruncus colihominis | |
| Jeotgalicoccus halotolerans* | Jeotgalicoccus halotolerans/ |
| | Jeotgalicoccus nanhaiensis |
| Butyricimonas faecihominis | |
| Pseudomonas gessardii* | Pseudomonas gessardii/Pseudomonas proteolytica |
| Parasutterella secunda | |
| Bacteroides finegoldii | |
| Barnesiella intestinihominis | |
| Blautia luti | |
| Citrobacter werkmanii* | Citrobacter werkmanii/Citrobacter freundii/ |
| | Citrobacter murliniae/Citrobacter braakii |
| Citrobacter gillenii* | Citrobacter gillenii/Citrobacter pasteurii |
| Oxalobacter formigenes | |
| Clostridium lactatifermentans | |
| Streptococcus gordonii | |
| Asaccharobacter celatus | |
| Howardella ureilytica | |
| Methylobacterium phyllosphaerae | |
| Blautia hansenii | |
| Coprobacillus cateniformis | |
| Planomicrobium chinense | |
| Parabacteroides faecis | |
| Alistipes inops | |
| Clostridium glycyrrhizinilyticum* | |
| Weissella confusa* | Weissella confusa/Weissella cibaria |
| Pelomonas aquatica | |
| Acidaminococcus fermentans | |
| Aggregatibacter segnis | |
| Romboutsia lituseburensis | |
| Sellimonas intestinalis | |
| Pyramidobacter piscolens | |
| Senegalimassillia anaerobia | |
| Clostridium butyricum | |
| Brachybacterium paraconglomeratum* | Brachybacterium paraconglomeratum/ |
| | Brachybacterium conglomeratum |
| Sutterella parvirubra | |
| Hungatella effluvii | |
| Kocuria carniohila* | Kocuria carniohila/Kocuria gwangalliensis |
| Microbacterium esteraomaticum* | Microbacterium esteraomaticum/ |
| | Microbacterium arabinogalactanolyticum |
| Aflipia broomeae | |
| Pseudochrobactrums | Pseudochrobactrums saccharolyticum/ |
| saccharolyticum* | Pseudochrobactrums asaccharolyticum/ |
| | Pseudochrobactrums lubricantis |
| Eubacterium oxidoreducens | |
| Bacteroides barnesiae | |
| Butyricimonas paravirosa | |
| Succinatimonas hippei | |
| Enterobacter xiangfangensis | |
| Enhydrobacter aerosaccus | |
| Mesorhizobium jarvisii* | Mesorhizobium jarvisii/ |
| | Mesorhizobium erdmanii/ |
| | Mesorhizobium opportunistum |
| Taonella mepensis | |
| Eubacterium sulci | |
| Clostridium citroniae | |
| Clostridium scindens | |
| Sphingomonas hankookensis | Sphingomonas hankookensis/ |
| | Sphingomonas panni |
| Bacteroides plebeius | |
| Eubacterium desmolans | |
| Micrococcus endophyticus | |
| Eubacterium contortum | Faecalicatena contorta comb. nov. |
| Dietzia cercidiphylli* | Dietzia cercidiphylli/Dietzia natronolimnaea |
| Desulfovibrio legallii | |
| Lactobacillus sanfranciscensis | |
| Kluyvera georgiana | |
| Pelomonas puraquae | |
| Olsenella scatoligenes | |
| Eubacterium dolichum | |
| Bacteroides coagulans | |
| Flavobacterium mizutaii | |
| Lactobacillus crispatus | |
| Treponema succinifaciens | |
| Lactonifactor longoviformis | |
| Brevundimonas aurantiaca | |
| Microbacterium sediminicola | |
| Phyllobacterium myrsinacearum | |
| Bacteroides sartorii | |
| Clostridium chartatabidum | |
| Kocuria palustris | |
| Anaerococcus murdochii | |
| Sphingobacterium nematocida | |
| Glutamicibacter protophormiae | |
| Prevotella buccalis | |
| Psychrobacter alimentarius | |
| Herbaspirillum huttiense | |
| Bradyrhizobium icense | |
| Collinsella intestinalis | |
| Desemzia incerta | |
| Cloacibacillus porcorum | |
| Comamonas denitrificans | |
| Brachybacterium ginsengisoli | |
| Bacteroides pectinophilus | |
| Vampirovibrio chlorellavorus | |
| Empedobacter falsenii | |
| Aggregatibacter aphrophillus | |
| Haemophilus sputorum | |
| Aeromonas punctata* | Aeromonas punctata/Aeromonas |
| | dhakensis/Aeromonas aquariorum |
| Methyloversatilis discipulorum* | Methyloversatilis discipulorum/ |
| | Methyloversatilis universalis |
| Paracoccus haeundaensis* | Paracoccus haeundaensis/Paracoccus |
| | marcusii/Paracoccus carotinifaciens |
| Brevundimonas bullata | |
| Phenylobacterium faslum | |
| Sphingomonas glacialis | |
| Rhodococcus degradans* | Rhodococcus degradans/Rhodococcus |
| | erythropolis/Rhodococcus |
| | qingshengii/Rhodococcus jialingiae |
| Slackia piriformis | |
| Staphylococcus captis | |
| Lactobacillus delbrueckii | |
| Cloacibacillus evryensis | |
| Alistipes putredinis | formerly Bacteroides putredinis |
| Bacteroides galacturonicus | |
| Leuconostoc citreum* | Leuconostoc citreum/Leuconostoc holzapfelii |
| Anaerostipes caccae | |
| Clostridium hiranonis | |
| Pseudomonas tolaasii* | Pseudomonas tolaasii/ |
| | Pseudomonas extremorientalis |
| Aquabacterium citratiphium | |
| Eubacterium xylanophilum | |
| Peptoniphilus duerdenii | |
| Psychrobacter namhaensis | |
| Arthrobacter russicus | |
| Pseudomonas parafulva* | |
| Moryella indoligenes | |
| Clostridium ventriculi | |
| Anaerococcus octavius | |
| Prevotella timonensis | |
| Bacteroides stercorirosoris | |
| Cytophaga hutchinsonii | |
| Vibrio natriegens | |
| Thauera terpenica | |
| Massilia norwichensis | |
| Brevibacterium samyangense | |
| Lactobacillus xiangfangensis | |
| Streptococcus peroris | |
| Sphingobacterium alimentarium | |
| Pseudocitrobacter anthropi* | Pseudocitrobacter anthropi/ |
| | Pseudocitrobacter faecalis |
| Obesumbacterium proteus | |
| Pseudomonas yamanorum* | Pseudomonas yamanorum/Pseudomonas minutum |
| Pseudomonas fragi* | Pseudomonas fragi/Pseudomonas deceptionensis |
| Psychrobacter maritimus | |
| Aquincola tertiaricarbonis | |
| Herminiimonas contaminans | |
| Uruburuella suis | |
| Pseudoxanthomonas broegbernensis | |
| Ensifer morelensis | |
| Caulobacter vibrioides* | Caulobacter vibrioides/Caulobacter segnis |
| Dietzia kunjamensis* | Dietzia kunjamensis/Dietzia maris |
| Leucobacter aridicollis | |
| Bacillus simplex* | Bacillus simplex/Bacillus |
| | muralis/Bacillus butanolivorans |
| Bacillus altitudinis* | Bacillus altitudinis/Bacillus |
| | stratosphericus/Bacillus aerophilus |
| Sporosatcina siberiensis | |
| Planococcus koreense | |
| Lactobacillus curvatus* | Lactobacillus curvatus/Lactobacillus |
| | sakei/Lactobacillus graminis |
| Lactobacillus agilis | |
| Weissella paramesenteroides | |
| Thermus scotoductus | |
| Murimonas intestini | |
| Stomatobaculum longum | |
| Anaerostipes rhamnosivorans | |
| Clostridium sardiniense | |
| Anaerofustis stercorihominis | |
| Paeniclostridium bifermentans | |
| Peptostreptococcus stomatis | |
| Ezakiekka peruensis | |
| Anaerococcus vaginalis | |
| Ruminococcus champanellensis | |
| Prochlorococcus marinus | |
| Prevotella dentalis* | Hallella seregens/Prevotella dentalis |
| Prevotella corporis | |
| Bacteroides faecichinchillae | |
| Pantoea rodasii | |
| Pseudomonas zhaodongensis* | Pseudomonas zhaodongensis/ |
| | Pseudomonas xanthomarina |
| Psychrobacter cibarius | |
| Acidovorax wautersii | |
| Variovorax paradoxus | |
| Hydrogenophaga pesudoflava* | Hydrogenophaga pesudoflava/ |
| | Hydrogenophaga luteola |
| Sphaerotilus natans | |
| Aquabacterium parvum | |
| Massilia varians | |
| Massilia haematophila | |
| Paraburkholderia fungorum* | Paraburkholderia fungorum/ |
| | Burkholderia insula/ |
| | Paraburkholderia phytofirmans |
| Neisseria oralis | |
| Pseudoxanthomonas japonensis* | Pseudoxanthomonas japonensis/ |
| | Pseudoxanthomonas mexicana |
| Stenotrophomonas rhizophila | |
| Thermomonas carbonis | |
| Rhizobium metallidurans | |
| Aureimonas ureilytica | |
| Bradyrhizobium ganzhouense* | Bradyrhizobium ganzhouense/ |
| | Bradyrhizobium guangdongense |
| Bradyrhizobium neotropicale | |
| Rhodoplanes serenus* | Rhodoplanes serenus/Rhodoplanes piscinae |
| Methylobacterium oryzae | |
| Methylobacterium adhaesivum* | Methylobacterium adhaesivum/ |
| | Methylobacterium gossopiicola/ |
| | Methylobacterium goesingense |
| Microvirga aerophila | |
| Bosea massiliensis | |
| Paracoccus sphaerophysae | |
| Devosia limi | |
| Devosia chinhatensis | |
| Devosia riboflavina | |
| Hyphomicrobium facile | |
| Sphingomonas pseudosanguinis | |
| Sphingomonas aestuarii | |
| Sphingomonas koreensis | |
| Sphingomonas xenophagum | |
| Sphingopyxis bauzanensis | |
| Novosphingobium capsulatum | |
| Belnapia soli | |
| Bifidobacterium faecale | |
| Microlunatus phosphovorus | |
| Arthrobacter globiformis* | Arthrobacter globiformis/ |
| | Arthrobacter pascens |
| Micrococcus cohnii | |
| Kocuria salsicia | |
| Pseudarthrobacter polychromogenes | |
| Georgenia satyanarayanai* | Georgenia satyanarayanai/ |
| | Oceanitalea nanhaiensis |
| Microbacterium testaceum | |
| Microbacterium deminutum* | Microbacterium deminutum/ |
| | Microbacterium pumilum/ |
| | Microbacterium saccharophilum |
| Microbacterium mitrae | |
| Microbacterium ginsengisoli | |
| Pseudoclavibacter helvolus* | Pseudoclavibacter helvolus/ |
| | Pseudoclavibacter terrae |
| Janibacter anophelis | |
| Bacillus flexus | |
| Bacillus galliciensis | |
| Chryseomicrobium amylolyticum | |
| Planococcus okeanokoites | |
| Listeria marthii | Listeria marthii/Listeria |
| | welshimer/Listeria innocua |
| Exiguobacterium aurantiacum* | Exiguobacterium aurantiacum/ |
| | Exiguobacterium mexicanum |
| Lactobacillus plantarum* | Lactobacillus plantarum/ |
| | Lactobacillus pentosus/ |
| | Lactobacillus herbarum |
| Lactobacillus vaginalis | |
| Lactobacillus mindensis | |
| Lactobacillus farciminis* | Lactobacillus farciminis/ |
| | Lactobacillus formosensis |
| Enterococcus sulfureus | |
| Carnobacterium inhibens | |
| Facklamia tabacinasalis | |
| Leuconostoc lactis | |
| Weissella viridescens | |
| Gemella asaccharolytica | |
| Deinococcus caeni | |
| Deinococcus reticulitermitis | |
| Deinococcus wulumuqiensis | |
| Clostridium saccharogumia | |

TABLE 4
List of Representative Mouse Intestinal Commensal Bacteria

| | Original Strain | | GenBank |
|---|---|---|---|
| Species | Designation | DSM No. | No. (16s) |
| Acetatifactor muris | CT-m2 | 23669 | HM989805 |
| Actinomyces bowdenii | ERD15F | 28589 | KR364730 |
| [Acutalibacter muris] | KB18 | 26090 | KR364749 |
| Akkermansia muciniphila | YL44 | 26127 | KR364731 |
| Alistipes sp. | CC-5826-WT-bac | 27924 | KF809885 |
| Anaerofustis stercorihominis | WHC-424-CC-1 | 28733 | KR364732 |
| Anaerosalibacter bizertensis | mAs-G9K2R3-TGB-98 | 29482 | KR364733 |
| Anaerotruncus colihominis | JM4-15 | 28734 | KR364734 |
| Bacillus licheniformis | D2411 | 28591 | KR364737 |
| Bacillus sp. | 17-NRDAF21 | 28590 | KR364736 |
| Bacillus subtilis | Amp-T18 | 28592 | KR364738 |
| Bacillus thermoamylovorans | Amp-choc 19 | 28625 | KR364739 |
| Bacteroides acidifaciens | JCM 10556 | 15896 | AB021164 |
| Bacteroides acidifaciens | JJM0207_2 | 100502 | KR364740 |
| [Bacteroides caecimuris] | I48 | 26085 | KR364741 |
| Bacteroides sartorii | A-C2-0 | 21941 | GQ456204 |
| Bacteroides vulgatus | 39a-cc-B-5824-ARE | 28735 | KR364743 |
| Bacteroides xylanisolvens | JJM0207_12 | 100015 | KR364742 |
| Bifidobacterium longum | SJ19 | 28736 | KR364744 |
| subsp. animalis | | | |
| Bifidobacterium longum | YL2 | 26074 | KR364745 |
| subsp. animalis | | | |
| [Blautia caecimuris] | SJ18 | 29492 | KR364746 |
| Blautia coccoides | A-C6-2 | 28737 | GQ456208 |
| Blautia coccoides | YL58 | 26115 | KR364747 |
| Blautia coccoides | PG-424-CC-9 | 29138 | KU196081 |
| Cellulosimicrobium cellulans | CD1-BL204R1 | 29131 | KR364748 |
| Clostridium bifermentans | G7K1R3-PYG-90 | 29423 | KR364757 |
| Clostridium bolteae | MT10-315-CC-8.2 | 29485 | KR364755 |
| Clostridium clostridioforme | YL32 | 26114 | KR364750 |
| Clostridium cochlearium | G7K4R3-PYG-96 | 29358 | KR364756 |
| Clostridium cocleatum | I50 | 1551 | Y18188 |
| Clostridium innocuum | A-C3-1 | 29132 | GQ456207 |
| Clostridium innocuum | I46 | 26113 | KR364751 |
| Clostridium mangenotii | G10-CCK1R4-PYG-100 | 29133 | KR364752 |
| Clostridium perfringens | MJJ0609-4-1 | 29991 | KR364753 |
| Clostridium ramosum | SRB509′-5-F-B | 29357 | KR364754 |
| Clostridium ramosum | PG-426-CC-8.1 | 29355 | KU196082 |
| Clostridium sordellii | MJJ0609-3-1 | 29974 | KR364762 |
| Clostridium sp. | PG-426-IM-1 | 100503 | KR364758 |
| Clostridium sporogenes | B-CC-163-3B | 29420 | KR364759 |
| Clostridium sporogenes | 2PG-426-CC-2 | 29422 | KU196083 |
| Clostridium symbiosum | SRB539-5-G-R | 29356 | KR364763 |
| [Cuneatibacter caecimuris] | BARN-424-CC-10 | 29486 | KR364760 |
| Enterococcus faecalis | BL6J-x-F-MMA-1-1 | 29488 | KR364768 |
| Enterococcus faecalis | KB1 | 32036 | KR364769 |
| Enterococcus gallinarum | JM-20 | 28564 | KR364766 |
| Enterococcus gallinarum | A-C2-0b | 28565 | GQ456213 |
| Enterococcus gallinarum | JJM0609-2-2 | 100110 | KU196084 |
| Enterococcus hirae | SB | 28619 | KR364767 |
| Enterorhabdus caecimuris | B7 | 21839 | DQ789120 |
| Enterorhabdus mucosicola | Mt1B8 | 19490 | AM747811 |
| [Enterorhabdus muris] | WCA-131-CoC-2 | 29508 | KR364735 |
| Escherichia coli | Mt1B1 | 28618 | AM944637 |
| [Extibacter muris] | 40cc-B-5824-ARE | 28560 | KR364761 |
| [Extibacter muris] | S-cc-6080-an-2-a | 28561 | KU196087 |
| Flavonifractor plautii | YL31 | 26117 | KR364773 |
| Flavonifractor plautii | WHC-424-CC-2 | 29136 | KU196086 |
| Flavonifractor plautii | mOs-SRB-10A-2011 | 29137 | KR364772 |
| [Flintibacter butyricus] | BLS21 | 27579 | KF447772 |
| [Frisingicoccus caecimuris] | PG-426-CC-1 | 28559 | KR364774 |
| Intestinimonas | SRB-521-5-I | 26588 | KC311367 |
| butyriciproducens | | | |
| [Irregularibacter muris] | 2PG-426-CC-4.2 | 28593 | KR364764 |
| Lactobacillus apodemi | IMCC1736 | 16748 | AJ871178 |
| Lactobacillus johnsonii | MIII-1B.7-2 | 100219 | KU196088 |
| Lactobacillus murinus | M-6244-3B | 28683 | KR364777 |
| Lactobacillus murinus | BARN-424-CC-5 | 28690 | KU196091 |
| Lactobacillus murinus | M5-8a | 100193 | KU196090 |
| Lactobacillus murinus | MJJ0609_7 | 100194 | KU196089 |
| Lactobacillus paracasei | 25 | 28675 | KR364779 |
| Lactobacillus reuteri | M-6220-5A | 28673 | KR364775 |
| Lactobacillus reuteri | I49 | 32035 | KR364776 |
| Lactobacillus reuteri | MI-2 | 100191 | KU196092 |
| Lactobacillus reuteri | JJM0207_8 | 100192 | KU196093 |
| Lactobacillus rodentium | MYMRS/TLU1 | 24759 | HQ851022 |
| Lactobacillus taiwanensis | ST-465-5-N | 28674 | KR364778 |
| Lactobacillus taiwanensis | MJJ0609_8 | 100220 | KU196094 |
| [Longibaculum muris] | MT10-315-CC-1.2-2 | 29487 | KR364765 |
| [Longicatena caecimuris] | PG-426-CC-2 | 29481 | KR364771 |
| [Longicatena caecimuris] | MT10P-315-CC-8 | 29490 | KU196085 |
| [Muribaculum intestinale] | YL27 | 28989 | KR364784 |
| [Muricomes intestini] | 2-PG-424-CC-1 | 29489 | KR364770 |
| Murimonas intestini | SRB-530-5-H | 26524 | KC311366 |
| Murimonas intestini | SRB-509-4-S-H | 27577 | KF601349 |
| Murimonas intestini | SRB-524-4-S-H | 27578 | KF601350 |
| Oceanobacillus caeni | Amp-T16 | 28620 | KR364780 |
| Paenibacillus lactis | pT2-260P | 28626 | KR364781 |
| Parabacteroides distasonis | SAB-131-CoC-3 | 29491 | KR364782 |
| Parabacteroides goldsteinii | BS-C3-2 | 29187 | GQ456205 |
| Parvibacter caecicola | NR06 | 22242 | GQ456228 |
| [Pasteurella caecimuris] | AA-424-CC-1 | 28627 | KR364783 |
| [Pasteurella caecimuris] | BIL2-426-CC-3 | 100775 | KU196095 |
| Pediococcus pentosaceus | JM-80 | 28628 | KR364785 |
| Proteus mirabilis | CD1-T1542 | 28629 | KR364787 |
| Pseudomonas sp. | 230G | 28630 | KR364786 |
| Staphylococcus lentus | WCA-CC-3-1 | 100111 | KR364789 |
| Staphylococcus warneri | JM-8 | 28567 | KR364790 |
| Staphylococcus xylosus | 33-ERD13C | 28566 | KR364788 |
| Stenotrophomonas maltophilia | pT2-440Y | 28631 | KR364791 |
| Streptococcus danieliae | ERD01G | 22233 | GQ456229 |
| Terrisporobacter glycolicus | CCK3R4-PYG-107 | 29186 | KR364793 |
| [Turicimonas muris] | YL45 | 26109 | KR364792 |

TABLE 5
List of Representative Human Potential Pathogenic
Commensal Bacteria (Pathobionts)

| Bacterial Species Name | Group Annotation |
|---|---|
| Parabacteroides distasonis | |
| Bacteroides caccae | |
| Dorea longicatena | |
| Bacteroides uniformis | |
| Coprococcus comes | |
| Roseburia hominis | |
| Haemophilus parainfluenzae | |
| Streptococcus vestibularis* | Streptococcus vestibularis/Streptococcus salivarius |
| Klebsiella pneumoniae | |
| Ruminococcus gnavus | |
| Bacteroides fragilis | |
| Ruminococcus torques | |
| Bilophila wadsworthia | |
| Pannonibacter phragmitetus | |
| Butyricimonas virosa | |
| Turicibacter sanguinis | |
| Granulicatella adiacens | |
| Bacteroides salyersiae | |
| Desulfovibrio piger | |
| Bacteroides nordii | Bacteroides nordii/Bacteroides salyersae |
| Comamonas kerstersii | |
| Collinsella aerofaciens | |
| Alistipes onderdonkii | |
| Fusobacterium mortiferum | |
| Clostridium leptum | |
| Bacteroides faecis | |
| Enterobacter cancerogenus* | Enterobacter cancerogenus/Enterobacter ludwigii |
| Clostridium bolteae | |
| Streptococcus australis* | Streptococcus australis/Streptococcus rubneri |
| Fusobacterium varium* | Fusobacterium varium/Fusobacterium ulcerans |
| Streptococcus parasanguinis | |
| Streptococcus infantis | |
| Clostridium disporicum | |
| Clostridium perfringens | |
| Clostridium ramosum | |
| Campylobacter concisus | |
| Parabacteroides goldsteinii | |
| Propionibacterium acnes | |
| Enterobacter aerogenes | |
| Raoultella ornithinolytica | |
| Clostridium symbiosum | |
| Delftia lacustris* | Delftia lacustris/Delftia tsuruhatensis |
| Clostridium paraputrificum | |
| Enterobacter asburiae | |
| Rhizobium radiobacter* | Beijerinckis fluminensis/Rhizobium radiobacter |
| Klebsiella oxytoca* | Klebsiella oxytoca/Klebsiella michiganensis |
| Streptococcus oralis* | Streptococcus oralis/Streptococcus mitis |
| Enterococcus faecium* | Enterococcus faecium/Enterococcus lactis |
| Clostridium lavalense | |
| Hungatella hathewayi | |
| Neisseria subflava* | Neisseria subflava/Neisseria flavescens |
| Parabacteroides gordonii | |
| Fusobacterium necrogenes | |
| Escherichia_Shigella group* | Escherichia/Shigella dysenteriae |
| Brevundimonas diminuta | |
| Clostridium aldenense | |
| Fusobacterium nucleatum | |
| subsp. animalis | |
| Kluyvera ascorbata | |
| Acidaminococcus intestini | |
| Streptococcus anginosus | |
| Clostridium innocuum | |
| Stenotrophomonas maltophilia* | Stenotrophomonas maltophilia/Stenotrophomonas |
| | geniculata/Stenotrophomonas hibiscicola/ |
| | Stenotrophomonas/pavanii/Stenotrophomonas beteli |
| Eggerthella lenta | |
| Eisenbergiella tayi | |
| Streptococcus gallolyticus | |
| Solobacterium moorei | |
| Parvimonas micra | |
| Granulicatella elegans | |
| Enterobacter cloacae | |
| Burkholderia arboris* | Burkholderia arboris/Burkholderia |
| | contanminans/Burkholderia lata |
| Campylobacter jejuni | |
| Fusobacterium nucleatum | |
| subsp. nucleatum | |
| Proteus mirabilis | |
| Gemella haemolysans | |
| Oribacterium sinus | |
| Kocuria polaris* | Kocuria polaris/Kocuria rosea |
| Peptostreptococcus anaerobuis | |
| Rahnella aquatilis | |
| Acinetobacter lwoffii | |
| Bacillus cereus* | Bacillus cereus/Bacillus toyonensis/ |
| | Bacillus thruringiensis/Bacillus anthracis |
| Staphylococcus hominis | |
| Streptococcus sanguinis | |
| Abiotrophia defectiva | |
| Haemophilus paraphrohaemolyticus | |
| Achromobacter insuavis* | Achromobacter insuavis/Achromobacter |
| | xylosoxidans |
| Brevibacterium sanguinis* | Brevibacterium sanguinis/Brevibacterium celere |
| Gemella sanguinis | |
| Fusobacterium nucleatum | |
| subsp. polymorphum | |
| Ralstonia pickettii | |
| Streptococcus downei* | Streptococcus downei/Streptococcus |
| | dentirousetti |
| Enterococcus faecalis | |
| Blautia coccoides* | Blautia coccoides/Blautia producta |
| Acinetobacter johnsonii | |
| Selenomonas noxia | |
| Staphylococcus gallinarum | |
| Staphylococcus cohnii | |
| Lactococcus garvieae* | Lactococcus garvieae/Lactococcus |
| | formosensis |
| Streptococcus mutans | |
| Finegoldia magna | |
| Alcaligenes faecalis | |
| Aerococcus viridans* | Aerococcus viridans/Aerococcus |
| | urinaeequi |
| Prevotella bivia | |
| Morganella morganii | |
| Sneathia sanguinegens | |
| Peptoniphilus harei | |
| Neisseria elongata | |
| Campylobacter ureolyticus | |
| Hafnia paralvei | |
| Arcobacter cryaerophilus | |
| Porphyromonas endodontalis | |
| Haemophilus pittmaniae | |
| Achromobacter spanius* | Achromobacter spanius/ |
| | Achromobacter marplatensis |
| Enterococcus casseliflavus* | Enterococcus casseliflavus/ |
| | Enterococcus gallinarum |
| Eubacterium infirmum | |
| Mogibacterium pumilum* | Mogibacterium pumilum/Migobacterium |
| | vescum/Migobacterium neglectum/ |
| | Migobacterium diversum |
| Eubacterium nodatum | |
| Prevotella baroniae | |
| Vibrio furnissii | |
| Acinetobacter berezineiae* | Acinetobacter berezineiae/ |
| | Acinetobacter guillouiae |
| Mycobacterium aubagnense | |
| Brochothrix thermosphacta* | Brochothrix thermosphacta/ |
| | Brochothrix campestris |
| Enterococcus durans* | Enterococcus durans/Enterococcus |
| | hirae/Enterococcus thailandicus |
| Streptococcus oligofermentans | |
| Streptococcus lutetiensis* | Streptococcus lutetiensis/ |
| | Streptococcus infantarius/ |
| | Streptococcus equinus |
| Gemella morbillorum | |
| Mycoplasma hominis | |
| Acinetobacter junii | |
| Lactobacillus iners | |
| Plesiomonas shigelloides | |
| Anaerococcus prevotii | |
| Prevotella disiens | |
| Comamonas terrigena | |
| Propionibacterium granulosum | |
| Prevotella bergensis | |
| Streptococcus intermedius | |
| Fusobacterium gonidiaformans | |
| Campylobacter gracilis | |
| Campylobacter hominis | |
| Clostridium tertium | |
| Porphyromonas bennonis | |
| Capnocytophaga granulosa | |
| Comamonas thiooxydans* | Comamonas thiooxydans/Comamonas |
| | testosteroni |
| Neisseria sicca | |
| Eikenella corrodens | |
| Cardiobacterium hominis | |
| Methylobacterium brachiatum* | Methylobacterium brachiatum/ |
| | Methylobacterium mesophilicum |
| Methylobacterium aminovorans* | Methylobacterium aminovorans/ |
| | Methylobacterium extorquens |
| Corynebacterium tuberculostearicum | |
| Corynebacterium kroppenstedtii | |
| Actinomyces naeslundii | |
| Microbacterium paraoxydans | |
| Microbacterium lacticum* | Microbacterium lacticum/ |
| | Microbacterium schleiferi |
| Atopobium parvulum | |
| Eggerthella sinensis | |
| Bacillus infantis | |
| Planococcus rifitoensis* | Planococcus rifitoensis/ |
| | Planococcus citreus |
| Staphylococcus saprophyticus* | Staphylococcus saprophyticus/ |
| | Staphylococcus xylosus |
| Staphylococcus pettenkoferi | |
| Staphylococcus sciuri | |
| Staphylococcus lentus* | Staphylococcus lentus/Staphylococcus |
| | vitulinus/Staphylococcus fleurettii |
| Macrococcus caseolyticus | |
| Campylobacter showae | |
| Cronobacter sakazakii* | Cronobacter sakazakii/Cronabacter |
| | malonaticus |
| Streptococcus pneumoniae* | Streptococcus pneumoniae/ |
| | Streptococcus pseudopneumoniae |
| Streptococcus suis | |
| Fusobacterium nucleatum | |
| subsp. vincentii | |

TABLE 6
List of Representative Opportunistic Commensal Bacteria that Cause Gut Diseases

| Gut Pathobionts | Inflammatory Condition | Mechanism |
|---|---|---|
| Bacteroides fragilis | IBD and colon cancer | Presence of antibiotic resistance genes |
| | | cepA, cfiA, and nim(A-E) and B. |
| | | fragilis toxin-mediated pro- |
| | | carcinogenesis inflammation |
| Adherent Invasive | Crohn's disease | Genetic susceptibility in NOD2, |
| E. coli (AIEC) | | mutation in autophagy genes ATG16L1 |
| | | and IRGM, and dysbiosis |
| Enterococcus faecalis | Ulcerative colitis | Dysbiosis and inflammation in IL-10 |
| | | deficient mice |
| Fusobacterium nucleatum | IBD and colorectal cancer | Dysbiosis and chronic inflammation |
| Clostridioides difficile | Pseudomembranous colitis | Secretion of toxins TcdA and TcdB |
| | | mediates disruption of epithelial barrier |
| Helicobacter hepaticus | Colitis and colon cancer | Th17 mediated inflammation |
| | in immunocompromised mice | |
| Segmented filamentous | Colitis and intestinal | Th17 mediated inflammation |
| bacteria | inflammation in mice | |
| Helicobacter pylori | Peptic ulcer disease and | Type IV secreted CagA protein |
| | gastritis | mediated inflammation in host |
| Proteus mirabilis | Crohn's disease | Intestinal inflammation through IL-18, |
| | | IL-1β and NOD-like receptor signaling |
| | | pathway |
| Klebsiella pneumoniae | Colitis in mice; primary | Pathobiont mediated disruption of |
| | sclerosing cholangitis | epithelial barrier and Th17 mediated |
| | | inflammation |
| Prevotellaceae and TM7 | Murine colitis | Perturbation in NLRP6 inflammasome |
| | | pathway |
| Vancomycin-resistant | Blood stream infection | Broad spectrum antibiotic use |
| Enterococcus spp. | following proliferation | downregulates RegIIIγ lectin |
| | in the gut | that kills Gram+ bacteria |
| Citrobacter rodentium | Murine colitis | Activation of signaling pathways by |
| | | proteins secreted by type III secretion |
| | | system |
| Klebsiella oxytoca | Antibiotic-associated | Enterotoxins tilimycin and tilivalline |
| | hemorrhagic colitis | mediate DNA adducts |

Some embodiments of the disclosure are directed to the apparatus described here for use in an immunoassay, such as but not limited to, a Western blot or enzyme-linked immunosorbent assay (ELISA) for diagnosing a subject with a B cell malignancy or a relapse or a recurrence of a B cell malignancy in a subject in remission of a B cell malignancy.

Additional embodiments are directed to an apparatus comprising at least one commensal bacteria antigen, or a plurality of commensal bacteria antigens, which can be used in any of the disclosed embodiments for diagnosing a B cell malignancy, such as a non-Hodgkin lymphoma (NHL). For example, the B cell malignancy is selected from the group consisting of: diffuse large B-cell lymphoma (DLBCL), follicular lymphoma (FL), Burkitt lymphoma (BL), mantle cell lymphoma (MCL), splenic marginal zone lymphoma (MZL), chronic lymphocytic leukemia (CLL), mucosa-associated lymphoid tissue lymphoma (MALT), multiple myeloma (MM), Waldenström macroglobulinemia (WM), hairy cell leukemia (HCL), and Hodgkin lymphoma (HL) as well as the precursor diseases of B cell malignancies such as monoclonal gammopathy of unknown significance (MGUS) and monoclonal B-cell lymphocytosis (MBL).

### Definitions

All terms used herein are intended to have their ordinary meaning in the art unless otherwise provided. All concentrations are in terms of percentage by weight of the specified component relative to the entire weight of the topical composition, unless otherwise defined.

As used herein, “a” or “an” shall mean one or more. As used herein when used in conjunction with the word “comprising,” the words “a” or “an” mean one or more than one. As used herein “another” means at least a second or more.

As used herein, all ranges of numeric values include the endpoints and all possible values disclosed between the disclosed values. The exact values of all half-integral numeric values are also contemplated as specifically disclosed and as limits for all subsets of the disclosed range. For example, a range of from 0.1% to 3% specifically discloses a percentage of 0.1%, 1%, 1.5%, 2.0%, 2.5%, and 3%. Additionally, a range of 0.1 to 3% includes subsets of the original range including from 0.5% to 2.5%, from 1% to 3%, from 0.1% to 2.5%, etc. It will be understood that the sum of all weight % of individual components will not exceed 100%.

By the term “consist essentially of”, “consists essentially of”, or “consisting essentially of”, it is meant that the components or ingredients include only the listed components along with the normal impurities present in commercial materials and with any other additives present at levels which do not affect the operation of embodiments of the disclosure, for instance at levels less than 5% by weight or less than 1% or even 0.5% by weight.

The term “effective amount” or “therapeutically effective amount” of an agent (e.g compounds having the structure of formula (I), etc.), as used herein, is that amount sufficient to effect beneficial or desired results, such as clinical results, and, as such, an “effective amount” depends upon the context in which it is being applied. In some embodiments, the compounds are administered in an effective amount for the treatment or prophylaxis of a disease, disorder or condition. In another embodiment, in the context of administering an agent that is an anticancer agent, an effective amount of an agent is, for example, an amount sufficient to achieve alleviation or amelioration or prevention or prophylaxis of one or more symptoms or conditions; diminishment of extent of disease, disorder, or condition; stabilized (i.e., not worsening) state of disease, disorder, or condition; preventing spread of disease, disorder, or condition; delay or slowing the progress of the disease, disorder, or condition; amelioration or palliation of the disease, disorder, or condition (e.g., cancer, etc.); and remission (whether partial or total), whether detectable or undetectable, as compared to the response obtained without administration of the agent.

Determining an effective amount of substance to be delivered can depend upon a number of factors including, for example, the chemical structure and biological activity of the substance, the age and weight of the subject, the precise condition requiring treatment and its severity, the treatment schedule or duration, and the route of administration. The term “administer” or “administration” includes the delivery of a composition or one or more pharmaceutically active ingredients to an animal, individual, or subject, such as a human, including, for example, any suitable methods that serve to deliver the composition or its active ingredients or other pharmaceutically active ingredients to the site in need of treatment. The route of administration can vary depending on various factors, such as, for example, the components or ingredients of the pharmaceutical composition or the type or nature of the pharmaceutically active and/or inert ingredients, the location in need of treatment, for example, the site of the potential or actual infection, the causative agent of the disease or disorder, e.g., microorganism involved in the disease or disorder, the severity of the disease or disorder, the age and physical condition of the individual, and the like, which a person of ordinary skill would understand to take into consideration. Some non-limiting examples of routes of administration of a composition or pharmaceutically active ingredient to an individual include oral (e.g., solid (e.g., tablet, capsule, caplet, powder), gel (e.g., gel cap), solution (e.g., syrup, drink)); intravenous (e.g., as a sterile solution free of particulate emboli and in a solvent system suitable for intravenous use); topical (e.g., as a cream, gel, lotion, or ointment); intrapulmonary, intraperitoneal, intramuscular, parenteral, sublingual, transdermal, intranasal, inhalation (e.g., nebulizer, spray), intraocular, intratracheal, intrarectal, vaginal, genicular skin, ocular (e.g., eye drops), ear drops, and the like. In the case of a pharmaceutical composition that contains more than one ingredient (active or inert), one way of administering such a composition is by mixing the ingredients (for example, in the form of a suitable unit dosage form, such as a tablet, capsule, solution, powder, etc.) and then the introduction of the dosage form. The ingredients can also be administered separately (simultaneously or sequentially, i.e., one after the other), provided that these ingredients reach favorable therapeutic levels so that the composition as a whole has a synergistic and/or desired effect.

The frequency of treatments depends upon a number of factors, such as the amount of the therapeutic composition administered per dose, the pharmacodynamics and pharmacokinetics of the therapeutic agent(s), as well as the health and history of the subject. The precise amount, number of doses, and timing of doses will be determined by the attending physician or veterinarian. Therapeutic compositions of the present disclosure can be administered to any subject or animal, including various mammals.

As used herein, the term “subject” refers to any organism to which a composition and/or compound in accordance with the disclosure may be administered, e.g., for experimental, diagnostic, prophylactic, and/or therapeutic purposes. Non-limiting examples of typical subjects or individuals include any animal (e.g., mammals such as dogs, cats, mice, rats, rabbits, sheep, pigs, horses, cows, cattle, non-human primates, and humans, etc.). A subject in need thereof is typically a subject for whom it is desirable to treat a disease, disorder, or condition as described herein. For example, a subject in need thereof can seek or be in need of treatment, require treatment, be receiving treatment, receive treatment in the future, or a human or animal that is under care by a trained professional for a particular disease, disorder, or condition.

Typically, the treatment of a disease, disorder, or condition (e.g., the conditions described herein such as cancer) is an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e., not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment.

### EXAMPLES

The following examples illustrate specific aspects of the instant description. The examples should not be construed as limiting, as the example merely provides specific understanding and practice of the embodiments and its various aspects.

### Example 1: Western Blot Analysis

### 1. Collection of Commensal Bacteria:

For the 7 purified strains of commonly found opportunistic commensal bacteria, the bacteria were grown in 50 ml or 100 ml of appropriate media (such as Luria broth or ATCC medium 1490) at appropriate oxygen conditions (aerobic or anaerobic) at 37° C. overnight with vigorous shaking. The cultured bacteria were harvested into appropriate centrifuge tubes and then spun down. The supernatants were thoroughly aspirated off without disturbing the bacterial pellets. The tubes were placed on ice before further processing.

For commensal bacteria of mouse small intestines and colons, the entire digestive tract from the stomach to the rectum of a euthanized mouse was dissected out. The small intestine was divided into three equal length sections corresponding to the duodenum (adjacent to the stomach), jejunum, or ileum (upstream of the colon). The colon was divided into two sections corresponding to the cecum and colon. For each section of the small intestine and colon, the intestinal contents were harvested and collected into a conical tube for the sections of small intestines and another conical tube for the sections of the colon. The intestinal contents were homogenized in PBS by vortexing vigorously for 5 minutes. The homogenized intestinal samples were centrifuged at 400 g for 5 min at 4° C. to remove the debris and mouse cells in the pellet. The supernatants of each tube were transferred into a new tube. The supernatants containing commensal bacteria were filtered through a 40 μm cell strainer to further remove cell clumps or large debris. The filtered samples were centrifuged at 8,000 g for 5 min at 4° C. to pellet commensal bacteria. The bacterial pellets were washed with PBS. The washed samples were centrifuged at 8,000 g for 5 min at 4° C. to pellet commensal bacteria again. The tubes were placed on ice before further processing.

### 2. Preparation of Commensal Bacterial Protein Antigens (e.g., Proteins, Glycoproteins, Lipoproteins):

The weights and volumes of bacterial pellets prepared in step 1 were measured. An appropriate volume of 2×SDS Sample Buffer (62.5 mM Tris, pH 6.8, 1% SDS, 15% glycerol, 2% β-mercaptoethanol and 0.005% bromophenol blue) was added into each tube to prepare protein concentrations at 10 μg/μl (or 10 mg/ml). Each tube with the bacteria pellet was sonicated in 2×SDS Sample Buffer with a Sonifier sufficient to solubilize bacterial proteins. The sonicated tubes were spun down briefly to collect the samples at the bottom of the tubes. Samples were boiled at 98° C. for 10 minutes in a heat block to denature the proteins. The tubes were chilled on ice. The boiled samples were stored in a −20° C. freezer before Western blot analysis.

### 3. Western Blot:

The commensal bacterial protein antigens prepared in step 2 were loaded into each well of 4-20% Gradient SDS-PAGE at 10 μl/sample/well (100 μg/sample/well) and the gels were run at 200 V for ˜35 minutes (or until the bromophenol blue dye of the samples migrated to a distance of ˜0.5 cm from the end or bottom of the gel) to separate the protein antigens according to their molecular weights.

The proteins from the SDS-PAGE gels were transferred to nitrocellulose membranes using an electroblotting apparatus at 400 mAmp (30-50 V) for 150 minutes at 4° C.

The membranes were washed with TBS-T for 3 times and 5 minutes per wash (3×5 min) on a rocker.

The membranes were blocked with 10% Non-fat dry milk/TBS-T at room temperature (RT) for 1 hour on a rocker. After blocking, the membranes were washed with TBS-T for 3×5 min.

Each membrane was incubated with a human plasma or mouse serum sample diluted at an appropriate dilution factor (1:500, 1:5000, or 1:50000) in sterile 2% BSA/TBS-T on a rocker at 4° C. overnight.

After incubation with the plasma or serum samples, the membranes were washed with TBS-T for 3×5 min.

Each membrane was incubated with 2 μg/ml of horse radish peroxidase (HRP)-conjugated goat anti-human IgG (H+L) (for human plasma samples) or HRP-conjugated goat anti-mouse IgG (H+L) (for mouse serum samples) secondary (2°) antibody (Ab) diluted in 2.5% Non-fat dry milk/TBS-T on a rocker at room temperature (RT) for 1 hour.

After incubation with the HRP-conjugated 2° Ab, the membranes were washed with TBS-T for 5×5 min.

A freshly prepared HRP chemiluminescent substrate was added to detect the HRP-labeled 2° Ab on each blot.

Chemiluminescence images of the immunoblots were acquired using a low-light imaging system or the blots were exposed to X-ray films for appropriate time periods to detect the chemiluminescence signals.

The Western blot data shown in FIGS. 1, 2, and 3 analyzed commensal bacterial protein samples, which were prepared from purified bacteria strains (Samples 1 to 7; all are commonly found opportunistic commensal bacteria) and intestinal commensal bacteria harvested from the small intestine (SI) and colons of M-Traf3-/- and TRAF3-sufficient wild type littermate control (LMC) mice, respectively.

Total protein lysates (directly lysed and sonicated in 2×SDS SB): protein concentration at 10 μg/μl (or 10 mg/ml)

For each bacterial protein sample, 10 μl/lane was loaded as follows:

### Dual Color Marker

1. Escherichia coli—DH5α (Gram−, Aerobic)
2. Escherichia coli—Stbl3 (Gram−, Aerobic)
3. Klebsiella pneumoniae (Gram−, Aerobic)
4. Staphylococcus epidermidis (Gram+, Aerobic)
5. Enterococcus faecalis (Gram+, Aerobic)
6. Clostridium bifermentans (Gram+; Anaerobic)
7. Bacteroides fragilis (Gram−, Anaerobic)
8. Pooled commensal bacteria harvested from LMC Small intestine (SI)
9. Pooled commensal bacteria harvested from LMC Colon
10. Pooled commensal bacteria harvested from M-Traf3-/- Small intestine (SI)
11. Pooled commensal bacteria harvested from M-Traf3-/- colon

### Blue Low Range Marker

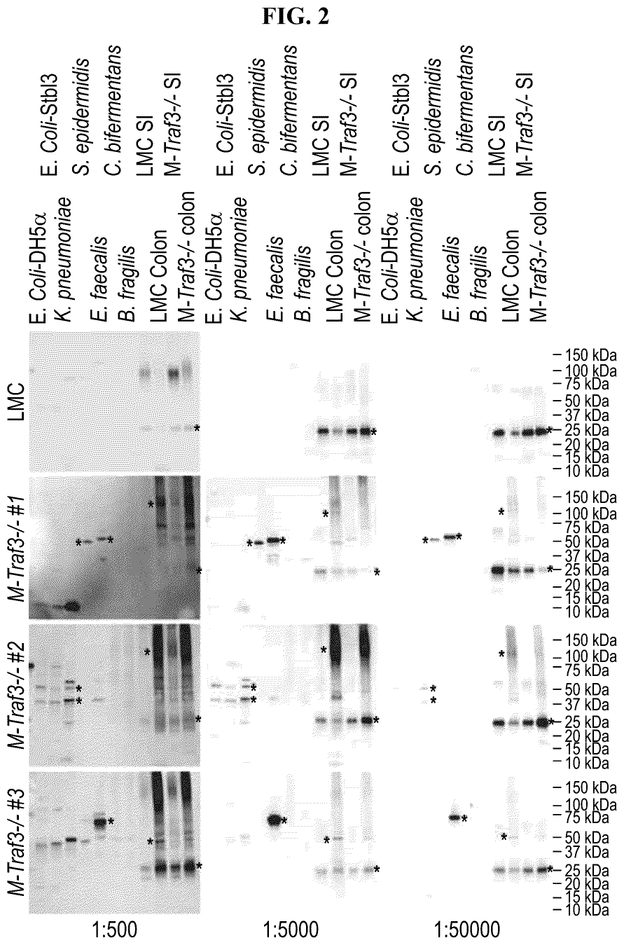

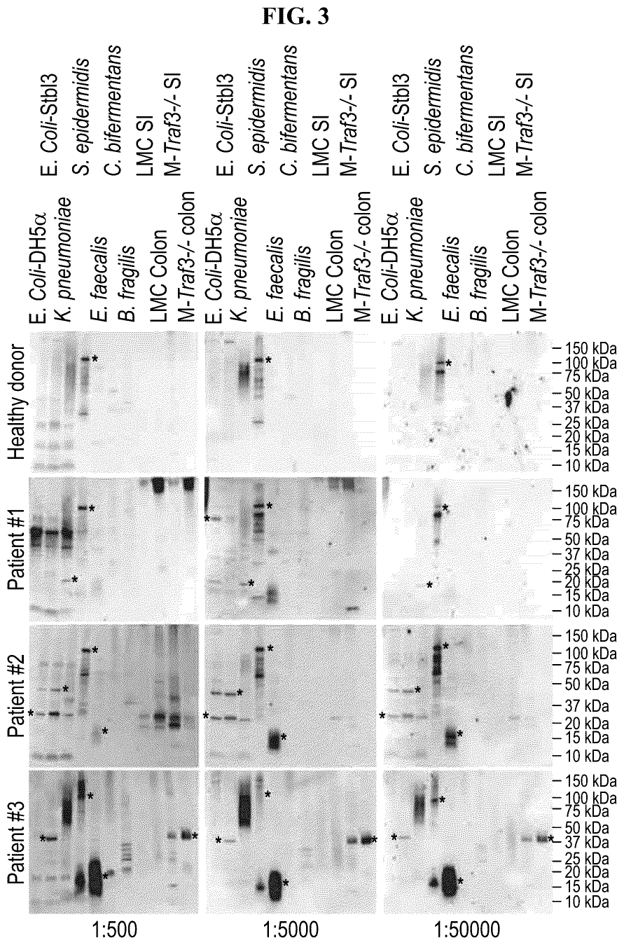

Primary (1°) antibodies (Ab): in FIG. 1, sera collected from M-Traf3-/- mice with spontaneous B lymphomas (DLBCL and FL) or TRAF3-sufficient wild type littermate control (LMC) mice were used as 1° Ab for Western blot analyses; in FIG. 2, sera collected from B-Traf3-/- mice with spontaneous B lymphomas (MZL and B1 lymphomas) or wild type LMC mice were used as 1° Ab for Western blot analyses; in FIG. 3, plasma samples of NHL patients or a healthy donor were used as 1° Ab for Western blot analyses.

The secondary (2°) antibody used for Western blot analyses was horseradish peroxidase (HRP)-goat anti-mouse IgG (H+L) for FIGS. 1 and 2; and HRP-conjugated goat anti-human IgG (H+L) for FIG. 3.

FIG. 1 used total protein lysates prepared from 7 purified strains of commonly found opportunistic commensal bacteria and intestinal commensal bacteria harvested from the small intestine (SI) and colons of M-Traf3-/- and TRAF3-sufficient littermate control (LMC) mice, respectively. Opportunistic commensal bacterial strains that were examined include E. coli—DH5α, E. coli—Stbl3, Klebsiella pneumoniae, Staphylococcus epidermidis (Gram+), Enterococcus faecalis (Gram+), Clostridium bifermentans (Gram+; anaerobic), and Bacteroides fragilis (anaerobic). Bacterial proteins were separated by 4-20% Gradient SDS-PAGE (BioRad) and blotted onto nitrocellulose membranes. After blocking with 10% milk/TBS-T, the membranes were probed with the serum samples at 1:500 (the left panel), 1:5000 (the middle panel), or 1:50000 (the right panel) dilutions, and then detected by HRP-conjugated goat anti-mouse IgG (H+L) secondary (2°) Ab. Serum samples shown include a TRAF3-sufficient littermate control (LMC) mice and 3 different M-Traf3-/- mice with spontaneous B lymphoma (DLBCL or FL) development. Molecular weight standards are shown at the right side of the immunoblots.

FIG. 2 used bacterial proteins separated by 4-20% Gradient SDS-PAGE (BioRad), blotted onto nitrocellulose membranes, and then probed with the serum samples at 1:500 (the left panel), 1:5000 (the middle panel), or 1:50000 (the right panel) dilutions. Serum samples shown included a TRAF3-sufficient littermate control (LMC) mice and 3 different B-Traf3-/- mice with spontaneous B lymphoma (MZL or B1 lymphoma) development.

FIG. 3 used bacterial proteins separated by 4-20% Gradient SDS-PAGE (BioRad), blotted onto nitrocellulose membranes, probed with the plasma samples at the indicated dilutions (1:500, 1:5000, or 1:50000), and then detected by HRP-conjugated goat anti-human IgG (H+L) secondary (2°) Ab. Plasma samples shown include a healthy donor control and 3 different patients with NHL.

### Example 2: ELISA Analysis

### 1. Collection of Commensal Bacteria:

For the 7 purified strains of commonly found opportunistic commensal bacteria, the bacteria were grown in 50 ml or 100 ml of appropriate media (such as Luria broth or ATCC medium 1490) at appropriate oxygen conditions (aerobic or anaerobic) at 37° C. overnight with vigorous shaking. The cultured bacteria were harvested into appropriate centrifuge tubes and then spun down. The supernatants were thoroughly aspirated off without disturbing the bacterial pellets. The tubes were placed on ice before further processing.

### 2. Preparation of Commensal Bacteria Antigen Lysates (e.g., Solubilized Proteins, Glycoproteins, Lipoproteins, Carbohydrates, Lipids, Nucleic Acids):

The weights and volumes of bacterial pellets prepared in step 1 were measured. An appropriate volume of freshly prepared Bacterial Resuspension buffer (20 mM Tris, pH 8, 150 mM NaCl, 1 mM DTT, 1× EDTA-free Mini-complete protease inhibitor cocktail) was added to each bacterial pellet at 300 μl/10 mg pellet. The bacteria pellet was thoroughly resuspended by vigorously vortexing for multiple rounds. When the bacteria pellet was thoroughly and completely resuspended, 1/100 volume of 100 mg/ml Lysozyme was added into each bacterial suspension (final concentration of Lysozyme: 1 mg/ml). Vortexed to mix well, and then the tubes were kept on ice for 20 min to lyse the bacteria. After 20 min incubation with Lysozyme, 1/10 volume of 10% Sarkosyl (final conc. of Sarkosyl: 1%) was added into the bacterial lysates. Each tube was vigorously vortexed to mix well. The suspended bacterial lysates were disrupted using an appropriately equipped sonicator for the suspended volume. Sonication was performed for 5 rounds at 25 pulses/round using an output appropriate for the suspended volume. The bacterial tubes were placed on ice during the sonication and were also rested on ice for 2 minutes between rounds of sonication. After sonication, the tubes were briefly spun down, and then the bacterial lysates were boiled at 98° C. for 10 min. After boiling, the tubes were immediately chilled on ice. The chilled tubes were centrifuged at 13,000 rpm for 20 min at 4° C. The supernatant from each tube was transferred to a new sterile tube. The concentration of bacterial proteins of each preparation was measured using the Bradford Protein Assay reagent. The cleared bacterial lysates were stored at −80° C. These were the bacterial lysate samples for ELISA.

For coating Immulon HB 96-well plates of ELISA, the clear bacterial lysates prepared in step 2 were diluted with appropriate volume of sterile PBS according to the concentration of bacterial proteins in each sample to prepare 200 μg bacterial antigens/ml in PBS for coating and mixed well by vortexing. The Immulon HB 96-well plates were coated with the diluted bacterial lysates at 100 μl/well (20 μg bacterial antigens/well).

The bacterial lysates (20 μg/100 μl/well) were incubated in the Immulon HB 96-well plates at 4° C. overnight to allow the binding of bacterial antigens onto the plates.

The coated plates were washed twice (2×) with PBS-T at 200 μl/well. After each wash, the wash buffer was discarded and the plates were blotted dry.

The plates were blocked with 200 μl/well of blocking buffer (1% BSA/PBS) at room temperature (RT) for 1 hour. After blocking, the plates were washed 3× with PBS-T at 200 μl/well. After each wash, the wash buffer was discarded and the plates were blotted dry.

Human plasma or mouse serum samples were diluted at appropriate dilution factors (1:100, 1:500, 1:2500, or 1:12500) in Blocking Buffer (1% BSA/PBS) and then were added into wells of the blocked plates at 100 μl/well. Duplicate 1:2 serial dilutions (100 μl/well) of a standard plasma or serum sample (from 1:40 up to 1:5120 dilutions) were included in each plate to determine the standard curve. Also included were at least 2 wells as blank wells in each plate by adding 100 μl/well of Blocking Buffer. The plates were incubated at 4° C. overnight.

After incubation with the plasma or serum samples, the plates were washed 3× with PBS-T at 200 μl/well. After each wash, the wash buffer was discarded and the plates were blotted dry.

10 μg/ml alkaline phosphatase (AP)-conjugated goat anti-human IgG (H+L) (for human plasma samples) or AP-conjugated goat anti-mouse IgG (H+L) (for mouse serum samples) 2° Ab was diluted in Blocking Buffer (1% BSA/PBS), added into wells of the ELISA plates at 100 μl/well, and incubated with the plates at room temperature (RT) for 1 hour.

After incubation with the AP-conjugated 2° Ab, each plate was washed 5× with PBS-T at 200 μl/well. For each wash of the first 4 washes, the wash buffer was discarded and the plates were blot dried. For the 5^{th} wash, after adding 200 μl of PBS-T, the wash buffer was not discarded until the substrates were thoroughly dissolved and ready for detection.

For each plate with 96 reactions, 1 mg/ml of the AP substrate solution was freshly prepared by dissolving two tablets of 5 mg phosphatase substrate (Sigma) in 10 ml of Substrate Buffer (1 M diethanolamine, pH 9.8, 0.5 mM MgCl₂). When the substrate solution was ready for detection, the 5^{th} wash buffer was discarded and then each plate was blot dried.

Substrate solution was added at 100 μl/well into each well of the plates. The reactions were developed at RT for a few minutes.

Each plate was read at 405 nm using a microplate reader. The plate was read when the most concentrated standard reached an OD405 of 1, and then read again when it approached an OD405 of 1.5, 2, and 2.5.

The reactions were stopped by adding 25 μl/well of 3N NaOH.

The commensal bacteria-specific antibody titers were calculated using the appropriate dilution factor of each sample and the standard curve of each plate.

The initial characterization of spontaneous B cell lymphoma development, including follicular lymphomas (FL) and diffuse large B-cell lymphomas (DLBCL), in aging myeloid cell-specific Traf3-deficient (M-Traf3-/-) mice was determined by Lalani et al. (J. Immunol., 194:334-348, 2015). The initial characterization of spontaneous B cell lymphoma development, including B1 lymphomas and marginal zone lymphomas (MZL), in B cell-specific Traf3-deficient (B-Traf3-/-) mice was illustrated by Moore et al. (Leukemia 26:1122-1127, 2012). The antibiotic treatment of these B cell lymphoma mouse models demonstrated that depletion of commensal bacteria prevented or inhibited B cell lymphoma development. See, FIGS. 4-5.

FIGS. 4A-4D show results of male and female mice (8-10-month-old) treated with antibiotics (1 mg/ml ampicillin, 1.6 mg/ml sulfamethoxazole and 0.32 mg/ml trimethoprim) in drinking water for 4 months. The health condition of the mice was monitored daily until moribund or 22-month-old (endpoint). Survival curves of TRAF3-sufficient littermate control mice (LMC) and M-Traf3-/- mice were generated using the Kaplan-Meier method. P value between the two curves of each graph was determined by the Mantel-Cox log-rank test. See, FIGS. 4A-4C.

FIGS. 5A-5D show results of male and female mice (2-3-month-old) treated with antibiotics (1 mg/ml ampicillin, 1.6 mg/ml sulfamethoxazole and 0.32 mg/ml trimethoprim) in drinking water for 4 months. Mouse health condition was monitored daily until moribund or 18-month-old (endpoint). Survival curves of TRAF3-sufficient littermate control mice (LMC) and B-Traf3-/- mice were generated using the Kaplan-Meier method. P value between the two curves of each graph was determined by the Mantel-Cox log-rank test. See, FIGS. 5A-5C.

Soluble lysates prepared from 6 (FIGS. 6A-6B) purified strains of commonly found opportunistic commensal bacteria, respectively, and used as the coating antigens to coat immunosorbent 96-well plates at 20 μg/well at 4° C. overnight. Coated plates were washed 3 times with PBS-T, and then blocked with 1% BSA/PBS at RT for 1 hour. The blocked plates were washed, and subsequently incubated with serum samples prepared from TRAF3-sufficient LMC mice of the M-Traf3 breeding colony (n=10), M-Traf3-/- mice (n=14) with spontaneous diffuse large B-cell lymphomas (DLBCL) or follicular lymphomas (FL) (FIG. 6A), TRAF3-sufficient LMC mice of the B-Traf3 breeding colony (n=8), B-Traf3-/- mice (n=8-10) with spontaneous marginal zone lymphomas (MZL) or B1 lymphomas, or LMC (n=8) and B-Traf3-/- mice (n=8) that received antibiotic treatment for 4 months (FIG. 6B). Dilution factors of serum samples tested in each plate include 1:100, 1:500, 1:2500, and 1:12500. Duplicate 1:2 serial dilutions of a standard serum sample (from 1:40 to 1:5120) were also included in each plate to determine the standard curve. After incubation with serum samples at 4° C. overnight, the plates were washed 3 times with PBS-T, and then incubated with 10 μg/ml AP-conjugated goat-anti-mouse IgG (H+L) 2° Ab at RT for 1 hour. The plates were subsequently washed 5 times with PBS-T and detected with 1 mg/ml of alkaline phosphatase (AP) substrates. Optical density at 405 nm (OD405) of each well in each plate was determined using a microplate reader with the SoftMax Pro 5.3 software. Commensal bacteria-specific antibody (Ig) titers were calculated using the appropriate dilution factor of each sample and the standard curve included in each plate by the SoftMax Pro 5.3 software. P values shown were analyzed by t test (FIG. 6A) or ANOVA (FIG. 6B).

The sera of M-Traf3-/- and B-Traf3-/- mice with spontaneous B lymphomas contained strikingly higher titers of antibodies specific for commensal bacterial antigens than tumor-free WT littermate control (LMC) mice, which was statistically significant (FIG. 6A; FIG. 6B). Furthermore, antibiotic treatment that substantially inhibited B lymphoma development and progression in B-Traf3-/- mice also significantly reduced the titers of antibodies specific for commensal bacterial antigens in aging B-Traf3-/- mice (FIG. 6B).

The high titers of antibodies specific for commensal bacterial antigens were produced by CD138+ plasmablasts/plasma cells that share the same immunophenotypic characteristics with splenic B lymphomas of B-Traf3-/- mice (FIG. 7A). Aging B-Traf3-/- mice with spontaneous B lymphomas contained CD138+ antibody-producing cells in the spleen and BM that share the immunophenotypic characteristics with splenic B lymphoma cells and were identified as B220+CD3−IgM^{hi}CD21−CD23−CD11b^{int}, which were not detected in aging LMC mice.

Moreover, sterile soluble commensal bacterial antigens were shown to induce B lymphoma cells of B-Traf3-/- mice to differentiate into CD138+ antibody-producing cells in ex vivo culture (FIG. 7B). Splenic B cells of aging LMC mice or young adult B-Traf3-/- mice and splenic B lymphoma cells of aging B-Traf3-/- mice were cultured ex vivo in the presence of 10 μg/ml of sterile soluble antigens of E. coli DH5α or 2 μg/ml agonistic anti-CD40 antibody plus 20 ng/ml IL-4, respectively, for 4 days. At 4 days after culture, cell surface expression of CD138, CD38, and IgM was determined by FACS analysis. FIG. 7B shows FACS profiles representative of 3 independent experiments. Approximately 5% of B-Traf3-/- splenic B lymphoma cells were induced to differentiate into CD138+ antibody-producing plasmablasts/plasma cells by sterile E. coli antigens, and ˜30% B lymphoma cells were differentiated into antibody-producing cells after treatment with anti-CD40 plus IL-4. Thus, the high titers of antibodies specific for commensal bacteria were produced by plasmablasts/plasma cells differentiated from the malignant B cell clones in these mice.

### Example 3: Analysis of Surface BCRs with Commensal Bacterial Antigens

The ability of the surface B cell antigen receptors (BCRs) expressed on splenic B lymphoma cells of B-Traf3-/- mice to react with commensal bacterial antigens was investigated (FIGS. 8A-8D). Splenocytes of aging LMC mice, young adult B-Traf3-/- mice, or aging B-Traf3-/- mice with spontaneous B lymphomas were cultured in the absence (Media) or presence of 10 μg/ml of sterile soluble antigens of E. coli DH5α (EC) for 10 min (10′) or 30 min (30′). Surface BCR and intracellular p-Btk levels on splenic B cells were determined by surface FACS and intracellular Phospho-Flow analyses, respectively.

IgM^{hi} splenic B lymphoma cells of 3 different individual B-Traf3-/- mice that contained high titers of antibodies against E. coli antigens were used in the ex vivo culture experiments. E. coli antigens induced rapid internalization of surface BCRs (IgM) in all 3 cases of splenic B lymphoma cells, which peaked within 10-30 min upon stimulation. E. coli antigens also robustly stimulated the proximal BCR signaling event, Bruton's Tyrosine Kinase (BTK) phosphorylation, in these B lymphoma cells. Follicular (FO) B cells were gated as B200+CD23+CD21^{int}, marginal zone (MZ) B cells were gated as B220+CD23^{int}CD21+, and double negative (DN) B cells were gated as B220+CD23−CD21− (FIG. 8A). The FACS data of FIG. 8B were representative of 3 independent experiments comparing the staining intensity of surface IgM (BCR) and intracellular phosphorylated Btk (p-Btk). Internalization of surface BCR, demonstrated by IgM down-regulation, and Btk phosphorylation induced by E. coli antigens were only detected in splenic B lymphoma cells of B-Traf3-/- mice, but not observed in different splenic B cell subsets of aging LMC mice or young adult tumor-free B-Traf3-/- mice. The FACS analyses of the geometric mean (GM) of surface IgM levels are shown in the media control (left column), E. coli DH5α (EC) for 10 minutes (center column), and EC for 30 minutes (right column) for each of the splenic B cell subsets of aging LMC mice or young adult B-Traf3-/- mice and splenic B lymphoma cells of aging B-Traf3-/- mice (FIG. 8C). The GM fold change of p-Btk levels in B lymphoma cells of aging B-Traf3-/- mice as determined by Phosph-Flow is illustrated in FIG. 8D. Together the results of FIGS. 8B-8D indicated that splenic B lymphoma cells of B-Traf3-/- mice express surface BCRs that recognize commensal bacterial antigens and that commensal bacterial antigens can engage BCR signaling to induce Btk phosphorylation. P values between each treatment group and the Media control were analyzed by ANOVA: **, P<0.01.

Based on the results of FIGS. 8A-8D, commensal bacterial antigens were seen as a major driver of B lymphoma development and progression by engaging BCR signaling in vivo. BTK is a clinically important target in many types of human B malignancies. The BTK inhibitor, ibrutinib, one of the best sold medicines in the world, exhibited moderate or minimal cytotoxic effects on cultured human malignant B cells in vitro and was highly effective in vivo in patients with B cell malignancies. This paradoxical phenomenon is explained here by the evidence that commensal bacterial antigens can induce BTK phosphorylation in vivo.

Commensal bacterial antigens-induced BCR internalization and BTK phosphorylation assays by FACS (FIGS. 8A-8D) were shown to be useful as a verification diagnostic method for human patients with B cell malignancies. Although still useful, the sensitivity of this method was not as high as that of the described Western blot and ELISA methods, because this FACS-based assay required the presence of at least 5% malignant B cells in patient blood samples or tumor specimens.

### Example 4: Analysis of Stimulation of Malignant B Cells by Commensal Bacterial Antigens

Sterile soluble commensal bacterial antigens were found to stimulate the survival and proliferation of human malignant B cells in culture. Soluble commensal bacterial antigens were extracted from 6 strains of common opportunistic commensal bacteria, including E. coli DH5α (EC), Klebsiella pneumoniae (KP), Staphylococcus epidermidis (SE), Enterococcus faecalis (EF), Clostridium bifermentans (CB), and Bacteroides fragilis (BF), and then sterilized by filtering through 0.2 μm. Human malignant B cell lines were cultured in vitro in the absence (Media) or presence of various concentrations (1:2 serial dilutions starting from 20 μg/ml) of sterile soluble antigens of commensal bacteria in 96-well plates for 72 h. Viable cells of each well were determined by MTT assay. Examined human malignant B cell lines include the Burkitt lymphoma (BL) cell lines, Daudi and Ramos; mantle cell lymphoma (MCL) cell lines, Z-138and JeKo-1; diffuse large B-cell lymphoma (DLBCL) cell lines, SU-DHL4 and SU-DHL6; and multiple myeloma (MM) cell lines, 8226 and KMS11. The graphs of FIG. 9 depict the peak growth-stimulatory effects of each cell line by each commensal bacterial strain-derived antigens as determined by MTT assay. The sterile soluble antigens of commensal bacteria stimulated the proliferation of human malignant B cells, including the BL cell lines, Daudi and Ramos; MCL cell lines, Z-138 and JeKo-1; DLBCL cell lines, SU-DHL-4 and SU-DHL-6; and MM cell lines, KMS11 and 8226 (FIG. 9). Results shown were obtained from 3 independent experiments with duplicate samples in each experiment (mean±SEM). P values between each treatment group and the Media control were analyzed by ANOVA: ns, P>0.05; *, P<0.05; **, P<0.01; ***, P<0.001.

### Example 5: Analysis of Protein Expression induced by Commensal Bacterial Antigens

Soluble commensal bacterial antigens were extracted from E. coli DH5α (EC) and Klebsiella pneumoniae (KP), and then sterilized by filtering through 0.2 μm (FIGS. 10A-10B). These commensal bacterial molecules induced the expression of chemokine receptors, adhesion molecules and immunosuppressive ligands important for migration and metastasis of malignant B cells. Cells from the Burkitt lymphoma (BL) cell line, Daudi; the mantle cell lymphoma (MCL) cell line, Z-138; and the multiple myeloma (MM) cell line, 8226, were cultured in the absence (Media) or presence of 10 μg/ml of sterile soluble antigens of E. coli DH5α (EC) or K. pneumoniae (KP) for 2 hours, 24 hours, or 72 hours. Total cellular RNA samples were prepared and reverse transcribed to generate cDNA. The transcript levels of CCR7, CD44, CD274 (encoding PD-L1), and CD273 (encoding PD-L2) were measured using gene-specific Taqman assays. Each PCR reaction also included the probe (VIC-labeled) and primers for bACTIN cDNA, which served as an endogenous control. Relative mRNA expression levels of each gene were analyzed using the comparative Ct method. The graphs of FIG. 10A depict the results of 3 independent experiments with duplicate reactions in each experiment (mean±SEM). In FIG. 10B, cells of the BL cell line, Daudi, were cultured in the absence (Media) or presence of 10 μg/ml of sterile soluble antigens of E. coli DH5α (EC) or K. pneumoniae (KP) for 72 hours, and then surface protein levels of CCR7, CD44, and PD-L1 were measured by FACS analysis. The graphs of FIG. 10B depict the results of the geometric mean (GM) for each surface protein obtained from 3 independent experiments (mean±SEM). P values were analyzed by ANOVA: *, significantly different from the Media control (P<0.05); **, very significantly different from the Media control (P<0.01); ***, highly significantly different from the Media control (P<0.001).

### Example 6: BCR and TLR Signaling Events Induced by Commensal Bacterial Antigens

Sterile commensal bacterial molecules were found to trigger both B cell receptor (BCR) and Toll-like receptor (TLR) signaling events, including the phosphorylation of: BTK (P-BTK), AKT (P-AKT), IKKE (P-IKKE), TBK1 (P-TBK1), and JNK1/2 (P-JNK1/2) in human malignant B cells. The soluble commensal bacterial antigens were prepared from E. coli DH5α (EC) and Klebsiella pneumoniae (KP), and then sterilized by filtering through 0.2 μm. Cells of a human malignant B cell line, Daudi, were cultured in the absence (Media) or presence of 10 μg/ml of sterile commensal bacterial antigens for the indicated time periods (10 minutes (min), 30 min, 60 min, 3 hours (h), 6h, 24h), and then total cellular proteins were prepared. BCR and TLR signaling events were determined by Western blot analyses (FIG. 11). Cellular proteins were separated by SDS-PAGE and immunoblotted for phosphorylated or total BTK, AKT, IKKε, TBK1, and JNK, followed by Actin control. Blots shown are representative of 3 independent experiments.

### Specific Embodiments

Non-limiting specific embodiments are described below, each of which is considered to be within the present disclosure.

Embodiment 1. A method, comprising:
(a) contacting at least one commensal bacterial antigen (e.g., protein; glycoprotein; lipoprotein; carbohydrate; lipid; nucleic acid) and a sample (e.g., blood; plasma; serum; ascites; pleural fluid; tissue fluid or interstitial fluid) from a subject suspected of or suffering from a B cell malignancy or relapse or recurrence of a B cell malignancy, thereby forming a contacted sample on an apparatus;
(b) incubating the contacted sample and a detection antibody (that binds to at least one immunoglobulin light chain of the subject species (κ or λ) and/or at least one immunoglobulin heavy chain of the subject species (IgG (e.g., IgG1, IgG2, IgG3, IgG4), IgA (IgA1, A2); IgM; IgD; IgE);
(c) detecting the detection antibody,
   wherein the detection antibody reflects the presence of at least one commensal bacteria-specific antibody in the subject;
(d) treating the subject comprising at least one commensal bacteria-specific antibody.

Embodiment 2. The method of embodiment 1, wherein an apparatus comprises the at least one commensal bacterial antigen.

Embodiment 3. The method of embodiment 2, wherein the at least one commensal bacterial antigen comprises a commensal bacterial protein (e.g., protein; glycoprotein; lipoprotein) or other molecule (e.g., carbohydrate; lipid; nucleic acid).

Embodiment 4. The method of embodiment 2, further comprising reducing background interference (e.g., blocking; washing) on the apparatus.

Embodiment 5. The method of embodiment 4, wherein reducing background interference occurs by blocking the apparatus before (a) contacting the at least one commensal bacterial antigen and a sample from the subject.

Embodiment 6. The method of embodiment 4, wherein reducing background interference occurs by washing the apparatus comprising the contacted sample before (b) incubating the contacted sample and the detection antibody, after (b) incubating the contacted sample and the detection antibody, or both before and after (b) incubating the contacted sample and the detection antibody.

Embodiment 7. The method of embodiment 1, wherein the detection antibody comprises specificity for at least one commensal bacteria-specific antibody.

Embodiment 8. The method of embodiment 1, wherein the detection antibody comprises specificity for at least one immunoglobulin light chain (κ or λ) of the subject.

Embodiment 9. The method of embodiment 1, wherein the detection antibody comprises specificity for at least one immunoglobulin heavy chain of the subject selected from the group consisting of: IgG (e.g., IgG1, IgG2, IgG3, IgG4), IgA1, IgA2, IgM, IgD, IgE, and any combination thereof.

Embodiment 10. The method of embodiment 1, wherein the detection antibody comprises a detectable label.

Embodiment 11. The method of embodiment 10, wherein the detectable label is selected from the group consisting of: an enzyme label, a fluorescent label, a radioactive label, and a biotin label.

Embodiment 12. The method of embodiment 11, wherein the detectable label is selected from the group consisting of: horseradish peroxidase (HRP), alkaline phosphatase (AP), β-galactosidase, glucose oxidase, glucose-6-phosphate dehydrogenase, β-N-acetylglucosaminidase, invertase, xanthine oxidase, biotin, Fluorescein Isothiocyanate (FITC), Tetramethylrhodamine-5-(and 6)-isothiocyanate (TRITC), Alexa Fluor® (AF) dye (e.g., AF 350, AF 405, AF 430, AF 488, AF 500, AF 514, AF 532, AF 546, AF 555, AF 568, AF 594, AF 610, AF 633, AF 635, AF 647, AF 660, AF 680, AF 700, AF 750, AF 790), Allophycocyanin (APC), phycoerythrin (PE), Brilliant violet (BV) dye (e.g., BV 421, BV 480, BV 510, BV 570, BV 605, BV 650, BV 711, BV 750, BV 785), Peridinin-chlorophyll-protein (PerCP), Pacific blue, Pacific orange, PE/Dazzle 594, APC Fire 750, Super Bright 436, Zombie Green, Texas Red, Rhodamine, and cyanine (Cy) dye (e.g., Cy2, Cy3, Cy5).

Embodiment 13. The method of embodiment 1, wherein (c) detecting occurs by immunoassay.

Embodiment 14. The method of embodiment 13, wherein the immunoassay is selected from the group consisting of: Western blotting and/or enzyme-linked immunosorbent assay (ELISA).

Embodiment 15. The method of embodiment 13, wherein the immunoassay is selected from the group consisting of: enzyme immunoassay (EIA), ELISA, fluoroimmunoassay (FIA), chemiluminescent immunoassay (CLIA), counting immunoassay (CIA), and radioimmunoassay (RIA).

Embodiment 16. The method of embodiment 1, wherein the commensal bacteria-specific antibodies of (c) comprises a titer of more than or equal to a 10-fold (e.g., 10 to 10⁵) increase as compared to a titer of a healthy subject (i.e., the healthy subject does not suffer from a B cell malignancy or an excess of commensal bacteria indicative of a B cell malignancy).

Embodiment 17. The method of embodiment 16, wherein the subject of (c) is diagnosed with a B cell malignancy.

Embodiment 18. The method of embodiment 1, wherein the treated subject (d) comprises a titer of commensal bacteria-specific antibodies of more than or equal to a 5-fold (e.g., 5 to 10⁵) increase as compared to a titer baseline of the subject after remission.

Embodiment 19. The method of embodiment 18, wherein the subject in remission has relapsed.

Embodiment 20. The method of embodiment 1, wherein the B cell malignancy is a non-Hodgkin lymphoma (NHL).

Embodiment 21. The method of embodiment 1, wherein the B cell malignancy is selected from the group consisting of: diffuse large B-cell lymphoma (DLBCL), follicular lymphoma (FL), Burkitt lymphoma (BL), mantle cell lymphoma (MCL), splenic marginal zone lymphoma (MZL), chronic lymphocytic leukemia (CLL), mucosa-associated lymphoid tissue lymphoma (MALT), multiple myeloma (MM), Waldenström macroglobulinemia (WM), hairy cell leukemia (HCL), Hodgkin lymphoma (HL), and precursor diseases of B cell malignancies (e.g., monoclonal gammopathy of unknown significance (MGUS); monoclonal B-cell lymphocytosis (MBL)).

Embodiment 22. The method of embodiment 1, wherein (d) treating comprises:
(i) administering at least one antibiotic in an effective amount sufficient to: reduce the at least one strain of commensal bacteria, alleviate a symptom of the B cell malignancy, palliate the B cell malignancy, slow the progression of the B cell malignancy, induce remission, or combinations thereof;
(ii) monitoring a titer of commensal bacteria-specific antibodies in the subject;
(iii) repeating the method of (a)-(d); or
(iv) combinations of (i)-(iii) thereof.

Embodiment 23. The method of embodiment 22, wherein administering at least one antibiotic comprises: oral administration, intravenous administration, intramuscular administration, or combinations thereof.

Embodiment 24. The method of embodiment 22, wherein administering at least one antibiotic comprises a treatment cycle comprising administering at least one antibiotic one or more times per day (e.g., 2 times, 3 times, 4, times).

Embodiment 25. The method of embodiment 24, wherein the treatment cycle comprises administering for at least 7 days consecutively (e.g., 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 50, 60, 70, 80, 90, 100, 110, 120, 130); 123 consecutive days or fewer (e.g., 122, 121, 120, 119, 118, 117, 116, 115, 114, 113, 112, 111, 110, 109, 108, 107, 106, 105, 104, 103, 102, 101, 100, 99, 98, 97, 96, 95, 94, 93, 92, 91, 90); or 7-130 consecutive days (e.g., 8-129; 9-128; 10-127; 11-126; 12-125; 13-124; 14-123; 15-122; 16-121; 17-120; 18-119; 19-118; 20-117; 21-116; 22-115; 23-114; 24-113; 25-112; 26-111; 27-110; 28-109; 29-108; 30-107; 31-106).

Embodiment 26. The method of embodiment 22, wherein the at least one antibiotic is selected (according to the subject's antibiotic tolerance and resistance profile) from the group consisting of: penicillin class, sulfonamide class, aminopyrimidine class, nitroimidazole class, glycopeptide class, quinolone class, lincosamide class, beta-lactam class, and combinations thereof.

Embodiment 27. The method of embodiment 22, wherein the at least one antibiotic is selected (according to the subject's antibiotic tolerance and resistance profile) from the group consisting of: ampicillin, sulfamethoxazole, trimethoprim, metronidazole, vancomycin, ciprofloxacin, levofloxacin, clindamycin, augmentin (i.e., amoxicillin/clavulanate), penicillin, cephalosporins, amoxicillin, amoxicillin/potassium clavulanate, streptomycin, gentamicin, neomycin/polymyxcin b/hydrocortisone, colistin/neomycin/thonzonium/hydrocortisone, nitrofurantoin, cephalexin, ciprofloxacin/dexamethasone, ciprofloxacin/hydrocortisone, ciprofloxacin, ofloxacin, ceftriaxone, fosfomycin, kanamycin, levofloxacin, imipenem/cilastatin, cefoxitin, and combinations thereof.

Embodiment 28. The method of embodiment 27, wherein the at least one antibiotic comprises ampicillin, sulfamethoxazole, and trimethoprim.

Embodiment 29. The method of embodiment 22, wherein the at least one antibiotic comprises at least two antibiotics (e.g., 3, 4, 5).

Embodiment 30. The method of embodiment 29, wherein the at least two antibiotics are administered together, separately, simultaneously (essentially), sequentially (essentially), or combinations thereof.

Embodiment 31. The method of embodiment 30, wherein the at least two antibiotics comprise three antibiotics.

Embodiment 32. The method of embodiment 31, wherein the three antibiotics comprise ampicillin, sulfamethoxazole, and trimethoprim.

Embodiment 33. The method of embodiment 32, wherein ampicillin is in an amount of 50 mg-1000 mg (e.g., 500 mg); and sulfamethoxazole and trimethoprim is in an amount ranging from 2.5 mg/kg-20 mg/kg (e.g., 8-10 mg/kg), where sulfamethoxazole and trimethoprim are in a ratio of 5:1 (e.g., 50 mg sulfamethoxazole and 10 mg trimethoprim; 100 mg sulfamethoxazole and 20 mg trimethoprim; 200 mg sulfamethoxazole and 40 mg trimethoprim; 400 mg sulfamethoxazole and 80 mg trimethoprim; 800 mg sulfamethoxazole and 160 mg trimethoprim).

Embodiment 34. The method of embodiment 32, wherein the ampicillin is administered 4 times per day.

Embodiment 35. The method of embodiment 32, wherein the ampicillin is administered once every 6 hours.

Embodiment 36. The method of embodiment 32, wherein sulfamethoxazole and trimethoprim are administered 2 times per day.

Embodiment 37. The method of embodiment 32, wherein sulfamethoxazole and trimethoprim are administered once every 12 hours.

Embodiment 38. The method of embodiment 32, wherein sulfamethoxazole and trimethoprim are administered together.

Embodiment 39. The method of embodiment 38, wherein ampicillin, sulfamethoxazole, and trimethoprim are administered simultaneously (essentially simultaneously).

Embodiment 40. The method of embodiment 22, further comprising subjecting the subject to an additional standard of care treatment selected from the group consisting of: chemotherapy, radiation therapy, immunotherapy, stem cell transplant, targeted drugs, surgery (e.g., surgical removal of lymphoma mass; splenectomy), and combinations thereof.

Embodiment 41. The method of embodiment 1, wherein the subject is a mammal (e.g., human, canine, feline, leporine, equine, bovine, murine, and the like).

Embodiment 42. The method of embodiment 1, wherein the apparatus (wherein the apparatus is configured for commensal bacterial antigen coating or protein deposition or conjugation) is selected from the group consisting of: a membrane (e.g., nitrocellulose; polyvinylidene fluoride (PVDF)), a multi-well plate (e.g., polystyrene, polyvinyl chloride), a bead, a chip (e.g., a microfluidic chip or a microchip), and a slide (wherein the apparatus is configured for commensal bacterial antigen coating or protein deposition or conjugation).

Embodiment 43. A method, comprising:
(a) contacting a plurality of commensal bacterial antigens (e.g., proteins; glycoproteins; lipoproteins; nucleic acids; carbohydrates; lipids) and a sample (e.g., blood; plasma; serum; ascites; tissue fluid or interstitial fluid) from a subject suspected of or suffering from a B cell malignancy or relapse or recurrence of a B cell malignancy, thereby forming a contacted sample on an apparatus;
(b) incubating the contacted sample and a detection enzyme-conjugated antibody (that binds to at least one immunoglobulin light chain of the subject species (κ or λ) or at least one immunoglobulin heavy chain of the subject species selected from the group consisting of: IgG (e.g., IgG1, IgG2, IgG3, IgG4), IgA1, IgA2, IgM, IgD, IgE, and any combination thereof);
(c) detecting the detection enzyme-conjugated antibody,
   wherein the detection enzyme-conjugated antibody reflects the presence of a plurality of commensal bacteria-specific antibodies in the subject; and
(d) treating the subject containing the plurality of commensal bacteria-specific antibodies.

Embodiment 44. The method of embodiment 43, wherein the apparatus comprises the plurality of commensal bacterial antigens, wherein the commensal bacterial antigens comprise the commensal bacterial antigens derived from a plurality of commensal bacteria.

Embodiment 45. The method of embodiment 43, further comprising reducing background interference (e.g., blocking; washing) on the apparatus.

Embodiment 46. The method of embodiment 45, wherein reducing background interference occurs by blocking the apparatus before (a) contacting the at least one commensal bacterial antigen and blood sample or other sample (e.g., plasma, serum, ascites, tissue fluid) from the subject.

Embodiment 47. The method of embodiment 45, wherein reducing background interference occurs by washing the apparatus comprising the contacted sample before (b) incubating the contacted sample and the detection antibody, after (b) incubating the contacted sample and the detection antibody, or both before and after (b) incubating the contacted sample and the detection antibody.

Embodiment 48. The method of embodiment 43, wherein the detectable enzyme-conjugated antibody comprises specificity for at least one commensal bacteria-specific antibody.

Embodiment 49. The method of embodiment 43, wherein the detectable enzyme-conjugated antibody comprises specificity for at least one immunoglobulin light chain (κ or λ) of the subject.

Embodiment 50. The method of embodiment 43, wherein the detectable enzyme-conjugated antibody comprises specificity for at least one immunoglobulin heavy chain of the subject selected from the group consisting of: IgG (e.g., IgG1, IgG2, IgG3, IgG4), IgA1, IgA2, IgM, IgD, IgE, and any combination thereof.

Embodiment 51. The method of embodiment 43, wherein the detectable enzyme-conjugated antibody comprises a detectable label.

Embodiment 52. The method of embodiment 51, wherein the detectable label is selected from the group consisting of: horseradish peroxidase (HRP), alkaline phosphatase (AP), β-galactosidase, glucose oxidase, glucose-6-phosphate dehydrogenase, β-N-acetylglucosaminidase, invertase, and xanthine oxidase.

Embodiment 53. The method of embodiment 43, wherein (d) detecting occurs by immunoassay.

Embodiment 54. The method of embodiment 53, wherein the immunoassay is selected from the group consisting of: Western blotting, enzyme-linked immunosorbent assay (ELISA), and combinations thereof.

Embodiment 55. The method of embodiment 53, wherein the immunoassay is selected from the group consisting of: ELISA, enzyme immunoassay (EIA), fluoroimmunoassay (FIA), chemiluminescent immunoassay (CLIA), flow cytometric immunoassay (e.g., FACS), counting immunoassay (CIA), and radioimmunoassay (RIA).

Embodiment 56. The method of embodiment 43, wherein the commensal bacteria-specific antibodies in the subject of (d) comprises a titer of more than or equal to a 10-fold (e.g., 10 to 10⁵) increase as compared to the titer of a healthy subject (i.e., the healthy subject does not suffer from a B cell malignancy or an excess of commensal bacteria indicative of a B cell malignancy).

Embodiment 57. The method of embodiment 56, wherein the subject of (d) is diagnosed with a B cell malignancy.

Embodiment 58. The method of embodiment 43, wherein the treated subject (e) comprises a titer of commensal bacteria-specific antibodies of more than or equal to a 5-fold (e.g., 5 to 10⁵) increase as compared to a titer baseline of the subject diagnosed in remission.

Embodiment 59. The method of embodiment 58, wherein the subject in remission has relapsed.

Embodiment 60. The method of embodiment 43, wherein the B cell malignancy is a non-Hodgkin lymphoma (NHL).

Embodiment 61. The method of embodiment 43, wherein the B cell malignancy is selected from the group consisting of: diffuse large B-cell lymphoma (DLBCL), follicular lymphoma (FL), Burkitt lymphoma (BL), mantle cell lymphoma (MCL), splenic marginal zone lymphoma (MZL), chronic lymphocytic leukemia (CLL), mucosa-associated lymphoid tissue lymphoma (MALT), multiple myeloma (MM), Waldenström macroglobulinemia (WM), hairy cell leukemia (HCL), and Hodgkin lymphoma (HL) as well as the precursor diseases of B cell malignancies such as monoclonal gammopathy of unknown significance (MGUS) and monoclonal B-cell lymphocytosis (MBL).

Embodiment 62. The method of embodiment 43, wherein (e) treating comprises:
(i) administering at least one antibiotic in an effective amount sufficient to: reduce the at least one strain of commensal bacteria, alleviate a symptom of the B cell malignancy, palliate the B cell malignancy, slow the progression of the B cell malignancy, induce remission, or combinations thereof;
(ii) monitoring a titer of commensal bacteria-specific antibodies in the subject;
(iii) repeating the method of (a)-(d); or
(iv) combinations thereof.

Embodiment 63. The method of embodiment 62, wherein administering at least one antibiotic comprises: oral administration, intravenous administration, intramuscular administration, or combinations thereof.

Embodiment 64. The method of embodiment 62, wherein administering at least one antibiotic comprises a treatment cycle comprising orally administering at least one antibiotic one or more times per day (e.g., 2 times, 3 times, 4, times).

Embodiment 65. The method of embodiment 64, wherein the treatment cycle comprises administering for at least 7 days consecutively (e.g., 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 50, 60, 70, 80, 90, 100, 110, 120, 130); 123 consecutive days or fewer (e.g., 122, 121, 120, 119, 118, 117, 116, 115, 114, 113, 112, 111, 110, 109, 108, 107, 106, 105, 104, 103, 102, 101, 100, 99, 98, 97, 96, 95, 94, 93, 92, 91, 90); 7-130 consecutive days (e.g., 8-129; 9-128; 10-127; 11-126; 12-125; 13-124; 14-123; 15-122; 16-121; 17-120; 18-119; 19-118; 20-117; 21-116; 22-115; 23-114; 24-113; 25-112; 26-111; 27-110; 28-109; 29-108; 30-107; 31-106).

Embodiment 66. The method of embodiment 62, wherein the at least one antibiotic is selected (according to the subject's antibiotic tolerance and resistance profile) from the group consisting of: penicillin class, sulfonamide class, aminopyrimidine, class, nitroimidazole class, glycopeptide class, quinolone class, lincosamide class, beta-lactam class, and combinations thereof.

Embodiment 67. The method of embodiment 62, wherein the at least one antibiotic is selected (according to the subject's antibiotic tolerance and resistance profile) from the group consisting of: ampicillin, sulfamethoxazole, trimethoprim, metronidazole, vancomycin, ciprofloxacin, levofloxacin, clindamycin, augmentin (i.e., amoxicillin/clavulanate), penicillin, cephalosporins, amoxicillin, amoxicillin/potassium clavulanate, streptomycin, gentamicin, neomycin/polymyxcin b/hydrocortisone, colistin/neomycin/thonzonium/hydrocortisone, nitrofurantoin, cephalexin, ciprofloxacin/dexamethasone, ciprofloxacin/hydrocortisone, ciprofloxacin, ofloxacin, ceftriaxone, fosfomycin, kanamycin, levofloxacin, imipenem/cilastatin, cefoxitin, and combinations thereof.

Embodiment 68. The method of embodiment 67, wherein the at least one antibiotic comprises ampicillin, sulfamethoxazole, and trimethoprim.

Embodiment 69. The method of embodiment 62, wherein the at least one antibiotic comprises at least two antibiotics (e.g., 3, 4, 5).

Embodiment 70. The method of embodiment 69, wherein the at least two antibiotics are administered together, separately, simultaneously (essentially), sequentially (essentially), or combinations thereof.

Embodiment 71. The method of embodiment 70, wherein the at least two antibiotics comprise three antibiotics.

Embodiment 72. The method of embodiment 71, wherein the three antibiotics comprise ampicillin, sulfamethoxazole, and trimethoprim.

Embodiment 73. The method of embodiment 72, wherein ampicillin is in an amount of 50 mg-1000 mg (e.g., 500 mg); and sulfamethoxazole and trimethoprim in an amount ranging from 2.5 mg/kg-20 mg/kg (e.g., 8-10 mg/kg), where sulfamethoxazole and trimethoprim are in a ratio of 5:1 (e.g., 50 mg sulfamethoxazole and 10 mg trimethoprim; 100 mg sulfamethoxazole and 20 mg trimethoprim; 200 mg sulfamethoxazole and 40 mg trimethoprim; 400 mg sulfamethoxazole and 80 mg trimethoprim; 800 mg sulfamethoxazole and 160 mg trimethoprim).

Embodiment 74. The method of embodiment 72, wherein the ampicillin is administered 4 times per day; and sulfamethoxazole and trimethoprim are administered 2 times per day.

Embodiment 75. The method of embodiment 74, wherein the ampicillin is administered once every 6 hours; and sulfamethoxazole and trimethoprim are administered once every 12 hours.

Embodiment 76. The method of embodiment 72, wherein sulfamethoxazole and trimethoprim are administered together.

Embodiment 77. The method of embodiment 76, wherein ampicillin, sulfamethoxazole, and trimethoprim are administered simultaneously (essentially simultaneously).

Embodiment 78. The method of embodiment 62, further comprising subjecting the subject to an additional standard of care treatment selected from the group consisting of: chemotherapy, radiation therapy, immunotherapy, stem cell transplant, targeted drugs, surgery, and combinations thereof.

Embodiment 79. The method of embodiment 43, wherein the subject is a mammal (e.g., human, canine, feline, leporine, equine, bovine, murine, and the like.).

Embodiment 80. The method of embodiment 43, wherein the apparatus is selected from the group consisting of: a membrane (e.g., nitrocellulose; polyvinylidene fluoride (PVDF)), a multi-well plate (e.g., polystyrene, polyvinyl chloride), a bead, a chip (e.g., a microfluidic chip or microchip), and a slide, wherein the apparatus is configured to enable or allow commensal bacterial antigen coating or protein deposition or conjugation.

Embodiment 81. An apparatus, comprising at least one commensal bacterial antigen (e.g., protein; glycoprotein; lipoprotein; carbohydrate; lipid; nucleic acid) attached thereto, for diagnosing a B cell malignancy or detecting a B cell malignancy relapse.

Embodiment 82. The apparatus of embodiment 81, wherein the apparatus is selected from the group consisting of: a membrane, a multi-well plate, a bead, a chip, and a slide.

Embodiment 83. The apparatus of embodiment 81, wherein the at least one commensal bacterial antigen comprises at least one commensal bacterial molecule (e.g., protein; glycoprotein; lipoprotein; nucleic acid; carbohydrate; lipid).

As various changes can be made in the above-described subject matter without departing from the scope and spirit of the present disclosure, it is intended that all subject matter contained in the above description, or defined in the appended embodiments and/or claims, be interpreted as descriptive and illustrative of the present disclosure. Many modifications and variations of the present disclosure are possible in light of the above teachings. Accordingly, the present disclosure is intended to embrace all such alternatives, modifications and variances which fall within the scope of the appended embodiments and/or claims.

All documents cited or referenced herein and all documents cited or referenced in the herein cited documents, together with any manufacturer's instructions, descriptions, product specifications, and product sheets for any products mentioned herein or in any document incorporated by reference herein, are hereby incorporated by reference, and may be employed in the practice of the disclosure.

What is claimed is:

1. A method, comprising:
(a) contacting at least one commensal bacterial antigen and a sample from a subject suspected of or suffering from a B cell malignancy or relapse or recurrence of a B cell malignancy, thereby forming a contacted sample on an apparatus;
(b) incubating the contacted sample and a detection antibody that binds to at least one immunoglobulin light chain of the subject species and/or at least one immunoglobulin heavy chain of the subject species;
(c) detecting the detection antibody,
wherein the detection antibody reflects the presence of at least one commensal bacteria-specific antibody in the subject; and
(d) treating the subject comprising at least one commensal bacteria-specific antibody.

2. The method of claim 1, wherein the apparatus comprises the at least one commensal bacterial antigen.

3. The method of claim 2, wherein the at least one commensal bacterial antigen comprises a commensal bacterial protein or other molecule.

4. The method of claim 2, further comprising reducing background interference on the apparatus.

5. The method of claim 1, wherein the detection antibody comprises specificity for at least one commensal bacteria-specific antibody.

6. The method of claim 1, wherein the detection antibody comprises specificity for at least one immunoglobulin light chain (κ or λ) of the subject.

7. The method of claim 1, wherein the detection antibody comprises specificity for at least one immunoglobulin heavy chain of the subject selected from the group consisting of: IgG, IgA1, IgA2, IgM, IgD, IgE, and any combination thereof.

8. The method of claim 1, wherein the detection antibody comprises a detectable label.

9. The method of claim 1, wherein (c) detecting occurs by immunoassay.

10. The method of claim 1, wherein the commensal bacteria-specific antibodies of (c) comprises a titer of more than or equal to a 10-fold increase as compared to a titer of a healthy subject.

11. The method of claim 10, wherein the subject of (c) is diagnosed with a B cell malignancy.

12. The method of claim 1, wherein the treated subject (d) comprises a titer of commensal bacteria-specific antibodies of more than or equal to a 5-fold increase as compared to a titer baseline of the subject after remission.

13. The method of claim 12, wherein the subject in remission has relapsed.

14. The method of claim 1, wherein the B cell malignancy is selected from the group consisting of:
diffuse large B-cell lymphoma (DLBCL), follicular lymphoma (FL), Burkitt lymphoma (BL), mantle cell lymphoma (MCL), splenic marginal zone lymphoma (MZL), chronic lymphocytic leukemia (CLL), mucosa-associated lymphoid tissue lymphoma (MALT), multiple myeloma (MM), Waldenström macroglobulinemia (WM), hairy cell leukemia (HCL), Hodgkin lymphoma (HL), non-Hodgkin lymphoma (NHL), and precursor diseases of B cell malignancies.

15. The method of claim 1, wherein (d) treating comprises:
(i) administering at least one antibiotic in an effective amount sufficient to: reduce the at least one strain of commensal bacteria, alleviate a symptom of the B cell malignancy, palliate the B cell malignancy, slow the progression of the B cell malignancy, induce remission, or combinations thereof;
(ii) monitoring a titer of commensal bacteria-specific antibodies in the subject;
(iii) repeating the method of (a)-(d); or
(iv) combinations of (i)-(iii) thereof.

16. The method of claim 15, wherein the at least one antibiotic is selected from the group consisting of: ampicillin, sulfamethoxazole, trimethoprim, metronidazole, vancomycin, ciprofloxacin, levofloxacin, clindamycin, augmentin, penicillin, cephalosporins, amoxicillin, amoxicillin/potassium clavulanate, streptomycin, gentamicin, neomycin/polymyxcin b/hydrocortisone, colistin/neomycin/thonzonium/hydrocortisone, nitrofurantoin, cephalexin, ciprofloxacin/dexamethasone, ciprofloxacin/hydrocortisone, ciprofloxacin, ofloxacin, ceftriaxone, fosfomycin, kanamycin, levofloxacin, imipenem/cilastatin, cefoxitin, and combinations thereof.

17. The method of claim 16, wherein ampicillin is in an amount of 50 mg-1000 mg; and
sulfamethoxazole and trimethoprim is in an amount ranging from 2.5 mg/kg-20 mg/kg, where sulfamethoxazole and trimethoprim are in a ratio of 5:1.

18. The method of claim 15, further comprising subjecting the subject to an additional standard of care treatment selected from the group consisting of: chemotherapy, radiation therapy, immunotherapy, stem cell transplant, targeted drugs, surgery, and combinations thereof.

19. A method, comprising:
(a) contacting a plurality of commensal bacterial antigens and a sample from a subject suspected of or suffering from a B cell malignancy or relapse or recurrence of a B cell malignancy, thereby forming a contacted sample on an apparatus;
(b) incubating the contacted sample and a detection enzyme-conjugated antibody that binds to at least one immunoglobulin light chain of the subject species or at least one immunoglobulin heavy chain of the subject species selected from the group consisting of: IgG, IgA1, IgA2, IgM, IgD, IgE, and any combination thereof;
(c) detecting the detection enzyme-conjugated antibody,
wherein the detection enzyme-conjugated antibody reflects the presence of a plurality of commensal bacteria-specific antibodies in the subject; and
(d) treating the subject containing the plurality of commensal bacteria-specific antibodies.

20. An apparatus, comprising at least one commensal bacterial antigen attached thereto, for diagnosing a B cell malignancy or detecting a B cell malignancy relapse according to claim 1.
